(19) United States
(12) Patent Application Publication
(10) Pub. No.: US 2023/0030606 A1
(43) Pub. Date: Feb. 2, 2023

(54) DEVICE AND METHOD OF USE FOR ASPIRATION SYSTEM RETRIEVERS

(71) Applicant: AngioDynamics, Inc., Latham, NY (US)

(72) Inventors: Kevin SWIFT, Hudson, MA (US); Greg EBERL, Acton, MA (US)

(21) Appl. No.: 17/869,687

(22) Filed: Jul. 20, 2022

Related U.S. Application Data
(60) Provisional application No. 63/223,847, filed on Jul. 20, 2021.

(51) Int. Cl. A61B 17/221

(52) U.S. Cl. CPC A61B 17/221; A61B 2017/00867

(57) ABSTRACT

Systems and methods for aspiration system retrievers that are configured to remove undesirable intravascular material. The retrievers can be utilized in connection with manually operated or automatic recirculation aspiration systems. The retrievers can be configured to transition between various configurations, including a collapsed configuration suitable for being advanced through a lumen of a catheter and an expanded configuration suitable for collecting or mechanically disrupting the undesirable intravascular material.

### PRIORITY

The present application claims priority to U.S. Provisional Patent Application No. 63/223,847, titled DEVICE AND METHOD OF USE FOR RETRACTOR BASKET, filed Jul. 20, 2021, which is hereby incorporated by reference herein in its entirety.

### BACKGROUND

Many of the most common and deadly diseases afflicting mankind result from or in the presence of undesirable material, most notably blood clots, in the blood vessels and heart chambers. Examples of such diseases include myocardial infarction, stroke, pulmonary embolism, deep vein thrombosis, atrial fibrillation, infective endocarditis, etc. The treatment of some of these conditions, which involve smaller blood vessels, such as myocardial infarction and stroke, has been dramatically improved in recent years by targeted mechanical efforts to remove blood clots from the circulatory system. Other deadly conditions, which involve medium to large blood vessels or heart chambers, such as pulmonary embolism (½ million deaths per year) or deep venous thrombosis (2-3 million cases per year) have not benefited significantly from such an approach. Present treatment for such conditions with drugs or other interventions is not sufficiently effective. As a result, additional measures are needed to help save lives of patients suffering from these conditions.

The circulatory system can be disrupted by the presence of undesirable material, most commonly blood clots, but also tumor, infective vegetations, and foreign bodies, etc. Blood clots can arise spontaneously within the blood vessel or heart chamber (thrombosis) or be carried through the circulation from a remote site and lodge in a blood vessel (thromboemboli).

In the systemic circulation, this undesirable material can cause harm by obstructing a systemic artery or vein. Obstructing a systemic artery interferes with the delivery of oxygen-rich blood to organs and tissues (arterial ischemia) and can ultimately lead to tissue death or infarction. Obstructing a systemic vein interferes with the drainage of oxygen-poor blood and fluid from organs and tissues (venous congestion) resulting in swelling (edema) and can occasionally lead to tissue infarction.

Many of the most common and deadly human diseases are caused by systemic arterial obstruction. The most common form of heart disease, such as myocardial infarction, results from thrombosis of a coronary artery following disruption of a cholesterol plaque. The most common causes of stroke include obstruction of a cerebral artery either from local thrombosis or thromboemboli, typically from the heart. Obstruction of the arteries to abdominal organs by thrombosis or thromboemboli can result in catastrophic organ injury, most commonly infarction of the small and large intestine. Obstruction of the arteries to the extremities by thrombosis or thromboemboli can result in gangrene.

In the systemic venous circulation, undesirable material can also cause serious harm. Blood clots can develop in the large veins of the legs and pelvis, a common condition known as deep venous thrombosis (DVT). DVT arises most commonly when there is a propensity for stagnated blood (long-haul air travel, immobility) and clotting (cancer, recent surgery, especially orthopedic surgery), DVT causes harm by (1) obstructing drainage of venous blood from the legs leading to swelling, ulcers, pain and infection and (2) serving as a reservoir for a blood clot to travel to other parts of the body including the heart, lungs (pulmonary embolism) and across a opening between the chambers of the heart (patent foramen ovale) to the brain (stroke), abdominal organs or extremities.

In the pulmonary circulation, the undesirable material can cause harm by obstructing pulmonary arteries, a condition known as pulmonary embolism. If the obstruction is upstream, in the main or large branch pulmonary arteries, it can severely compromise total blood flow within the lungs and therefore the entire body, resulting in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide. If the obstruction is further downstream, it can cut off the blood flow to a smaller portion of the lung, resulting in death of lung tissue or pulmonary infarction.

The presence of the undesirable material within the heart chambers can cause harm by obstructing flow or by serving as a reservoir for emboli to other organs in the body. The most common site for obstruction within the heart is in the heart valves. Infective vegetations, a condition known as endocarditis, can cause partial obstruction to flow across a valve before destroying the valve. Patients with prosthetic valves, especially mechanical valves, are particularly prone to valve thrombosis and obstruction. The heart chambers are the most common source of emboli (cardioemboli) to the systemic circulation, including stroke. Emboli tend to arise from areas that are prone to stagnation of blood flow under pathologic conditions. The left atrial appendage in patients with atrial fibrillation is prone to thrombosis, as well as the left ventricular apex in patients with acute myocardial infarction or dilated cardiomyopathy. Infected vegetations or thrombi on the heart valves are also common sources of emboli. Undesirable material such as blood clots and infected vegetations can reside in the chambers of the right heart (atrium and ventricle), often associated with prosthetic material such as pacemaker leads or long-term indwelling catheters.

The most effective treatment for conditions resulting from the presence of blood clots or other undesirable materials within the circulation is, of course, to stabilize or eliminate the material before it has embolized. Alternatively, if obstruction to flow has already occurred but before the obstruction has caused permanent harm (infarction, shock, death), the material can be eliminated by utilizing biologic or mechanical means.

Biologic treatments involve the delivery of agents to the material, which either dissolve the material or, at a minimum, stabilize it until the body can eliminate it. In the case of infective vegetations, antimicrobial agents can, over time, decrease the chances of embolization. In the case of blood clots, the agents include 1) anticoagulant agents (heparin, warfarin, etc.) which prevent propagation of blood clots; and 2) more potent thrombolytic agents (streptokinase, urokinase, tPA, etc,) which actively dissolve clots. The agents are usually delivered systemically, i.e., into a peripheral or central vein and allowed to circulate throughout the body. Thrombolytic agents can also be delivered through a catheter directly to the blood clot which can increase its effectiveness by increasing local concentrations but this does not completely eliminate the absorption into systemic circulation throughout the body.

Thrombolytic agents have been shown to increase survival in patients with hemodynamically significant pulmonary embolism as documented by echocardiographic evidence of right ventricular strain. The use of thrombolytic agents is the standard of care in this subgroup of patients with a high 20-25% early mortality. They are commonly used to dissolve clots in other blood vessels including arteries to the heart, abdominal organs and extremities.

There are two primary disadvantages to thrombolytic agents. First, every cell in the body is exposed to the agent which can lead to serious and often life threatening bleeding complications in remote areas such as the brain and stomach. The risk of major bleeding complications can be as high as 25% and the risk of often fatal bleeding into the brain can go up to 3%. Second, blood clots undergo a process called organization where the soft gel-like red/purple clot is transformed into a firmer, whitish clot by the cross-linking of proteins such as fibrin. Organized clots are much less amenable to treatment with thrombolytic agents. Thromboemboli, such as pulmonary emboli, can contain a significant amount of organized clots since the thrombus frequently developed at its original site (e.g., the deep veins of the legs) over a long period of time prior to embolizing to the remote site (e.g., the lungs).

Mechanical treatments involve the direct manipulation of the material to eliminate the obstruction. This can involve aspiration, maceration, and compression against the vessel wall, or other types of manipulation. The distinct advantage of mechanical treatment is that it directly attacks the offending material and eliminates the vascular obstruction independent of the specific content of the offending material. Mechanical treatments, if feasible, can usually prove to be superior to biologic treatments for vascular obstruction. Procedural success rates tend to be higher. The best example of this advantage is in the treatment of acute myocardial infarction. Although thrombolytic therapy has had a major impact on the management of patients with myocardial infarction, this option is now relegated to a distant second choice. The clear standard of care today for an acute myocardial infarction is an emergency percutaneous coronary intervention during which the coronary artery obstruction is relieved by aspiration, maceration or balloon compression of the offending thrombus. This mechanical approach has been shown to decrease the amount of damaged heart tissue and improve survival relative to the thrombolytic biological approach.

Mechanical treatment, however, has played a limited role in the removal of blood clots found in larger blood vessels such as pulmonary arteries and heart chambers. Surgical pulmonary embolectomy involves opening the pulmonary artery and removing the offending clot under direct vision. This operation has been performed for nearly 100 years, but did not become practical until the introduction of the heart lung machine. Even then, it was generally relegated to a salvage procedure in moribund patients in whom all other options had been exhausted because of the inherent danger in the surgery and the recovery period. While surgical pulmonary embolectomy is very effective in completely evacuating pulmonary emboli whether soft-fresh and firm-organized clot, it is an invasive procedure.

Recent data has shown that the early outcomes with surgical pulmonary embolectomy are excellent, at least as good as thrombolytic treatment, as long as the procedure is performed in a timely fashion before the patient becomes very ill or suffers a cardiac arrest. The long term outcomes of patients surviving surgical pulmonary embolectomy have always been very good. Although these data have generated a renewed interest in performing surgical pulmonary embolectomy, its use remains limited because of the invasiveness of the procedure. Although minimally invasive approaches have been described, the standard procedure requires a 20-25 cm incision through the sternal bone and placing the patient on cardiopulmonary bypass (the heart-lung machine).

Catheter-based removal of blood clots from larger blood vessels (e.g., pulmonary arteries) and heart chambers has had limited success, at least compared to smaller blood vessels (e.g., coronary arteries). Catheter pulmonary embolectomy, where the pulmonary emboli are removed percutaneously using one of several techniques, has been around for nearly 30 years but few patients currently receive these therapies. These techniques can be subdivided into three categories. With fragmentation thrombectomy, the clot is broken into smaller pieces, most of which migrate further downstream, decreasing the central obstruction but resulting in a “no-reflow” phenomenon. It is sometimes used in combination with thrombolytics which preclude their use as an alternative to thrombolytics. With the rheolytic thrombectomy, high velocity saline jets create a Venturi effect and draw the fragments of the clot into the catheter. Finally, the aspiration techniques draw the clot into a catheter via suction. With a Greenfield embolectomy, the catheter with the attached clot is repeatedly drawn out of the vein. All of these techniques rely on catheters which are small compared to the size of the clots and blood vessels. Their limited success is likely related to their inability to achieve a complete en-bloc removal of the material without fragmentation.

The experience with catheter-based treatment of deep venous thrombus has also had limited success. The operator must use relatively small catheters to remove or break up large amounts of well embedded clot. This procedure is therefore time-consuming, inefficient and ultimately not very effective in removal of the whole clot.

It is clear that all of the therapeutic options available to patients with clots or other undesirable material in medium or large blood vessels, such as those with pulmonary embolism, have serious limitations. Anticoagulation only limits propagation of clot, it does not remove it. Thrombolytic therapy is not targeted, carries a real risk of major bleeding, and is not very effective in firm/organized clots. Catheter embolectomy uses technology developed for small blood vessels, does not scale well to material residing in medium and large vessels or heart chambers, and thus is not very effective. Surgical embolectomy is highly effective but highly invasive. There is a real need for a direct mechanical treatment that is as effective as surgical embolectomy but can be performed using endovascular techniques.

There is a need in the art for an improved systems and methods to endovascularly remove undesirable material from a patient's body.

### SUMMARY

The present disclosure is directed to retrievers for use in conjunction with or as components of aspiration systems that are adapted to remove thrombi and other undesirable intravascular material (UIM). In particular, the retrievers described herein can be used to remove adherent UIM, i.e., UIM that are adhering to the internal walls of the vessel or other anatomical structure.

In some embodiments, there is provided a retriever for use with an aspiration system for removal of undesirable intravascular material (UIM), the aspiration system comprising a catheter, the retriever comprising: a plurality of struts that are interconnected to define a mesh structure, the mesh structure comprising a first section and a second section connected at an inversion point; wherein: the mesh structure is configured to transition between a collapsed configuration, an expanded configuration, and a deployed configuration, in the collapsed configuration, the mesh structure comprises an outer diameter less than or equal to an internal diameter of a lumen of the catheter such that the mesh structure is configured to be movable through the lumen, the mesh structure is configured to transition from the collapsed configuration to the expanded configuration in response to exiting the catheter, in the expanded configuration, the outer diameter of the mesh structure is greater than the outer diameter in the collapsed configuration, in the expanded configuration, the mesh structure is configured such that application of a longitudinal force thereto causes the second section to invert at the inversion point and collapse into the first section, thereby forming a semi-spherical shape defining the deployed configuration, and the semi-spherical shape is configured to receive and hold the UIM.

In some embodiments, there is provided a retriever for use with an aspiration system for removal of UIM, the aspiration system comprising a catheter, the retriever comprising: a plurality of struts that are interconnected to define a mesh structure, the mesh structure comprising a helical shape terminating at a pointed end; wherein the mesh structure comprises an outer diameter less than or equal to an internal diameter of a lumen of the catheter such that the mesh structure is configured to be movable through the lumen.

In some embodiments, there is provided a retriever for use with an aspiration system for removal of undesirable intravascular material (UIM), the aspiration system comprising a catheter, the retriever comprising: a plurality of struts that are interconnected to define a mesh structure, the mesh structure comprising a semi-spherical shape; wherein: the mesh structure is configured to transition between a collapsed configuration and an expanded configuration, in the collapsed configuration, the mesh structure comprises an outer diameter less than or equal to an internal diameter of a lumen of the catheter such that the mesh structure is configured to be movable through the lumen, in the expanded configuration, the outer diameter of the mesh structure is greater than the outer diameter in the collapsed configuration, and the semi-spherical shape is configured to receive and hold the

### FIGURES

FIG. 1 shows a perspective view of a manual aspiration system, in accordance with an embodiment of the present disclosure.

FIG. 2 shows a perspective view of an aspiration device shown in a resting position, in accordance with an embodiment of the present disclosure.

FIG. 3 shows a perspective view of the aspiration device of FIG. 2 in a fully retracted position, in accordance with an embodiment of the present disclosure.

FIG. 4 shows a side view of an aspiration device in a resting position shown in partial cross-section, in accordance with an embodiment of the present disclosure.

FIG. 5 shows a side view of the aspiration device of FIG. 4 in a fully retracted position, in accordance with an embodiment of the present disclosure.

FIGS. 6A-6C show partial side views of an aspiration device, in accordance with an embodiment of the present disclosure. FIG. 6A shows the device in a resting position. FIG. 6B shows the device in a partially retracted position. FIG. 6C shows the device in a fully retracted position.

FIG. 7A shows a side view of a suction cannula/sheath subassembly, in accordance with an embodiment of the present disclosure.

FIG. 7B shows a perspective view of an aspiration system indicating the movement of undesirable material therethrough, in accordance with an embodiment of the present disclosure.

FIG. 8A shows a diagram of a reinfusion aspiration system for removing undesirable intravascular material, in accordance with an embodiment of the present disclosure.

FIG. 8B shows the aspiration system of FIG. 8A deployed within a subject, in accordance with an embodiment of the present disclosure.

FIG. 9 shows a perspective view of a retriever in an expanded configuration, in accordance with an embodiment of the present disclosure.

FIG. 10 shows a diagram of the retriever of FIG. 9 in the expanded configuration, in accordance with an embodiment of the present disclosure.

FIG. 11 shows a view of the retriever of FIG. 9 in a collapsed configuration, in accordance with an embodiment of the present disclosure.

FIG. 12 shows a side view of the retriever of FIG. 9 in a deployed configuration, in accordance with an embodiment of the present disclosure.

FIG. 13 shows an end view of the retriever of FIG. 9 in a deployed configuration, in accordance with an embodiment of the present disclosure.

FIG. 14A shows a diagram of a delivery device for delivering the retriever shown in FIG. 9, in accordance with an embodiment of the present disclosure.

FIG. 14B shows a diagram of the delivery device shown in FIG. 14A, in accordance with an embodiment of the present disclosure.

FIG. 14C shows a sectional view of the delivery device of FIG. 14A alone line 14-14, in accordance with an embodiment of the present disclosure.

FIG. 14D shows a perspective view of an alternative embodiment of a delivery device for delivering a retriever, in accordance with an embodiment of the present disclosure.

FIG. 14E shows a reverse perspective view of the first guide bracket of the delivery device shown in FIG. 14D, in accordance with an embodiment of the present disclosure.

FIG. 14F shows a reverse perspective view of the second guide bracket of the delivery device shown in FIG. 14D, in accordance with an embodiment of the present disclosure.

FIG. 14G shows a perspective view of the delivery device shown in FIG. 14D coupled to a handle assembly, in accordance with an embodiment of the present disclosure.

FIG. 15A shows a perspective view of a handle assembly for a catheter delivery device, in accordance with an embodiment of the present disclosure.

FIG. 15B shows an overheard, partially cutaway view of the handle assembly of FIG. 15A, in accordance with an embodiment of the present disclosure.

FIG. 15C shows a side view of the handle assembly of FIG. 15A, in accordance with an embodiment of the present disclosure.

FIG. 15D shows a side, partially cutaway view of the handle assembly of FIG. 15A, in accordance with an embodiment of the present disclosure.

FIG. 15E shows a reverse perspective view of the handle assembly of FIG. 15A, in accordance with an embodiment of the present disclosure.

FIG. 15F shows an exploded view of the handle assembly of FIG. 15A, in accordance with an embodiment of the present disclosure.

FIG. 15G shows a view of the retriever in the deployed configuration and the corresponding handle assembly control position, in accordance with an embodiment of the present disclosure.

FIG. 15H shows a view of the retriever in the collapsed configuration and the corresponding handle assembly control position, in accordance with an embodiment of the present disclosure.

FIG. 151 shows a view of the retriever in the expanded configuration and the corresponding handle assembly control position, in accordance with an embodiment of the present disclosure.

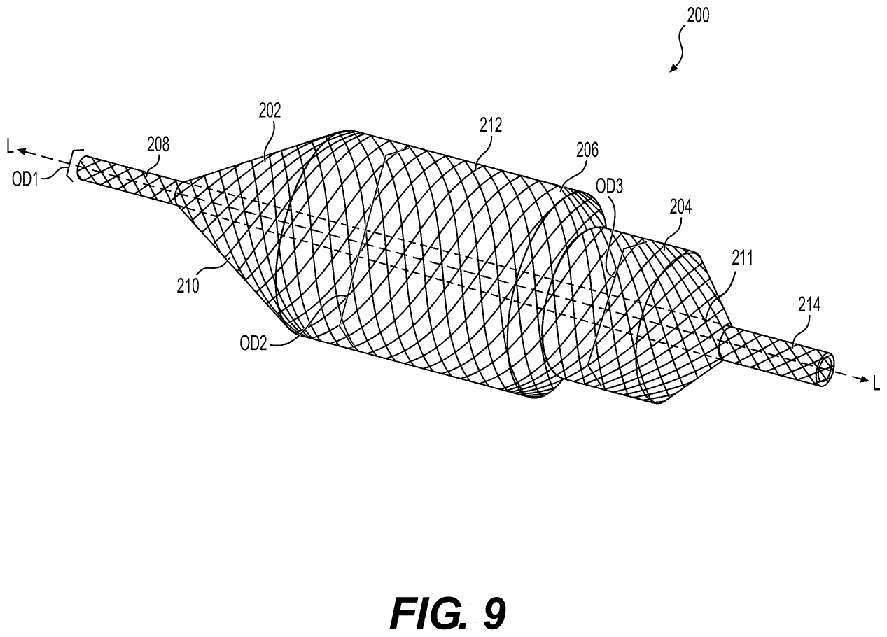

FIG. 16A shows a perspective view of the retriever of FIG. 9 engaged with a suction cannula, in accordance with an embodiment of the present disclosure.

FIG. 16B shows a reverse perspective view of the retriever of FIG. 9 engaged with a suction cannula, in accordance with an embodiment of the present disclosure.

FIG. 17 shows a side view of an invertible retriever having a uniform diameter, in accordance with an embodiment of the present disclosure.

FIG. 18A shows a perspective view of an invertible retriever having three sections, in accordance with an embodiment of the present disclosure.

FIG. 18B shows a side view of the multi-section invertible retriever shown in FIG. 18A, in accordance with an embodiment of the present disclosure.

FIG. 19 shows a view of a retriever including a spring section, in accordance with an embodiment of the present disclosure.

FIG. 20 shows a view of a retriever including reinforcement struts, in accordance with an embodiment of the present disclosure.

FIG. 21A shows a perspective view of a deployable reinforcement strut assembly in its undeployed configuration, in accordance with an embodiment of the present disclosure.

FIG. 21B shows a perspective view of the deployable reinforcement strut assembly shown in FIG. 21A in its deployed configuration, in accordance with an embodiment of the present disclosure.

FIG. 21C shows a diagram of the deployable reinforcement strut assembly of FIG. 21A positioned within a collapsed retriever, in accordance with an embodiment of the present disclosure.

FIG. 21D shows a diagram of the deployable reinforcement strut assembly of FIG. 21A positioned within an expanded retriever in the expanded configuration, in accordance with an embodiment of the present disclosure.

FIG. 21E shows a sectional view of the deployable reinforcement strut assembly of FIG. 21A positioned within an expanded retriever, in accordance with an embodiment of the present disclosure.

FIG. 21F shows an axial view of the deployable reinforcement strut assembly of FIG. 21A positioned within an expanded retriever, in accordance with an embodiment of the present disclosure.

FIG. 21G shows a reverse perspective view of the deployable reinforcement strut assembly of FIG. 21A positioned within an expanded retriever, in accordance with an embodiment of the present disclosure.

FIG. 21H shows a cutaway perspective view of the deployable reinforcement strut assembly of FIG. 21A positioned within an expanded retriever, in accordance with an embodiment of the present disclosure.

FIG. 21I shows a view of an alternative embodiment of a reinforcement device in a collapsed configuration, in accordance with an embodiment of the present disclosure.

FIG. 21J shows a view of the reinforcement device of FIG. 211 in an intermediate configuration, in accordance with an embodiment of the present disclosure.

FIG. 21K shows a view of the reinforcement device of FIG. 211 in an expanded configuration, in accordance with an embodiment of the present disclosure.

FIG. 22 shows a view of a retriever including a reinforcing internal helical structure, in accordance with an embodiment of the present disclosure.

FIG. 23 shows a perspective view of an expandable, noninvertible retriever, in accordance with an embodiment of the present disclosure.

FIG. 24 shows a reverse perspective view of the retriever of FIG. 23, in accordance with an embodiment of the present disclosure.

FIG. 25A shows a perspective view of an alternative embodiment of an expandable, noninvertible retriever, in accordance with an embodiment of the present disclosure.

FIG. 25B shows a side view of the expandable, noninvertible retriever of FIG. 25A, in accordance with an embodiment of the present disclosure.

FIG. 26A shows a diagram of a reinforcing element for use with the expandable, noninvertible retrievers shown in FIGS. 23-25B, in accordance with an embodiment of the present disclosure.

FIG. 26B shows a diagram of a connector for use with the reinforcing element shown in FIG. 26A, in accordance with an embodiment of the present disclosure.

FIG. 26C shows an embodiment of the loop portion of the connector shown in FIG. 26B, in accordance with an embodiment of the present disclosure.

FIG. 26D shows an alternative embodiment of the loop portion of the connector shown in FIG. 26B, in accordance with an embodiment of the present disclosure.

FIG. 26E shows another alternative embodiment of the loop portion of the connector shown in FIG. 26B, in accordance with an embodiment of the present disclosure.

FIG. 26F shows an axial view of the reinforcing element shown in FIG. 26A, in accordance with an embodiment of the present disclosure.

FIG. 26G shows a diagram of a distal portion of a retriever, in accordance with an embodiment of the present disclosure.

FIG. 27A shows a diagram of the reinforcing element shown in FIG. 26A partially assembled within a retriever, in accordance with an embodiment of the present disclosure.

FIG. 27B shows a diagram of the reinforcing element shown in FIG. 26A connected to the tethers for the embodiment of the retriever shown in FIGS. 25A and 25B, in accordance with an embodiment of the present disclosure.

FIG. 27C shows the reinforcing element shown in FIG. 26A in a partially collapsed configuration, in accordance with an embodiment of the present disclosure.

FIG. 27D shows the reinforcing element shown in FIG. 26A in a fully collapsed configuration, in accordance with an embodiment of the present disclosure.

FIG. 28 shows a diagram of the reinforcing element shown in FIGS. 26A-D connected to the retriever tethers, in accordance with an embodiment of the present disclosure.

FIG. 29 shows a view of a helical retriever, in accordance with an embodiment of the present disclosure.

FIG. 30 shows a flow chart of a process for capturing undesirable intravascular material using the retriever assemblies described herein, in accordance with an embodiment of the present disclosure.

FIG. 31 shows a flow chart of a process for mechanically disrupting undesirable intravascular material using the retriever assemblies described herein, in accordance with an embodiment of the present disclosure.

FIG. 32 shows a flow chart of a process for capturing or mechanically disrupting undesirable intravascular material using the retriever assemblies described herein in combination with continuous reinfusion aspiration systems, in accordance with an embodiment of the present disclosure.

### DETAILED DESCRIPTION

This disclosure is not limited to the particular systems, devices and methods described, as these may vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “device” is a reference to one or more devices and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

As used herein, the term “undesirable intravascular material” (UIM) refers to intravascular debris including, but not limited to, thrombus, embolus, clot, vegetative growth, infected vegetative growth (such as endocarditis), pulmonary embolism, tumor, arterial clots, undesirable material trapped in dialysis grafts and/or stents, and other undesirable natural and/or unnatural foreign bodies to be removed from a subject's body.

As used herein, the term “en bloc” refers to entirely, wholly, and/or without significant fragmentation.

As used herein, the terms “suction force” and/or “vacuum force” refer to the negative pressure created by removing air from a space creating a pressure differential resulting in the force that a vacuum exerts upon the UIM. A drive force refers to the pressure differential generated by the device that exerts a force upon the UIM.

As used herein, the term “differential pressure” refers to the difference in pressure between two given points. Positive pressure refers to the pressure at a first point that is greater than pressure at a second point. Negative pressure refers a pressure at a first point that is lower than pressure at a second point.

As used herein, the term “vacuum” refers to a differential pressure, including decreases in pressure (i.e., negative pressure) below atmospheric pressure and increases in pressure (i.e., positive pressure) above atmospheric bidirectional differential pressure. For example, a vacuum or negative pressure for the suction force ranges from −11 psi to −14.7 psi and a positive pressure for the driving force ranges from +1 psi to +10 psi (i.e., the range of the return spring force).

As used herein, the term “trigger pull cycle” refers to the combined retraction or compression and release of the trigger assembly. Further, “fully retracted” refers to the maximum distance of travel for the trigger assembly starting from a rest position and/or deactivated state. Still further, “partially retracted” refers to any distance between the trigger assembly at a rest position and/or deactivated state and a full retraction of the trigger assembly, i.e., some distance less than the possible maximum distance of travel for the trigger assembly starting from a rest position and/or deactivated state.

Target vessels, treatment sites, or target areas include, but are not limited to, systemic venous circulation (e.g., inferior vena cava and/or superior vena cava, pelvic veins, leg veins, neck and arm veins); arterial circulation (e.g., aorta or its large and medium branches); heart chambers, such as in the left heart (e.g., the left ventricular apex and left atrial appendage), in the right heart (e.g., right atrium and right ventricle), or on its valves; small blood vessels; medium blood vessels; large blood vessels; iliofemoral vein; peripheral vasculature; and/or the pulmonary circulation (e.g., pulmonary veins and/or pulmonary arteries). In some embodiments, other treatment sites or target areas could include other nonvascular tubular structures, such as ducts or any other avascular tubular tissue. In some embodiments, other treatment sites or target areas could include, pacemaker leads, stents, or other artificial implanted medical devices.

This disclosure relates to devices and methods for minimally invasive removal of UIM from a vessel or other hollow anatomical structure of a subject. In particular, the disclosure relates to retrievers for use in conjunction with or as components of aspiration systems, wherein the retrievers are particularly adapted to facilitate the removal of adherent UIM. In some applications, the retrievers may be used to remove UIM en bloc; however, in other applications, the retrievers may be used to fragment or otherwise mechanically disrupt the UIM (e.g., when used in conjunction with a continuous suction and reinfusion aspiration system).

### Aspiration Systems

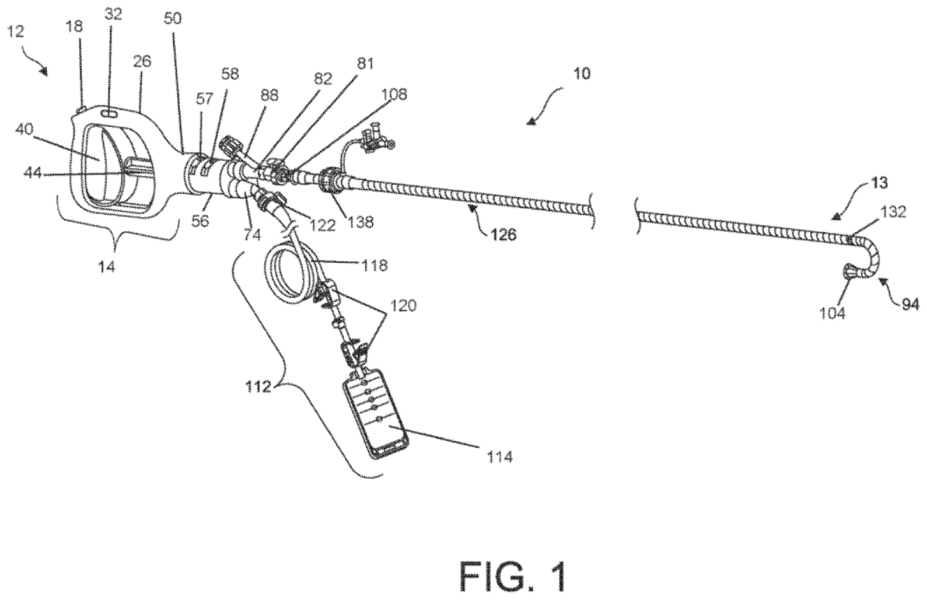

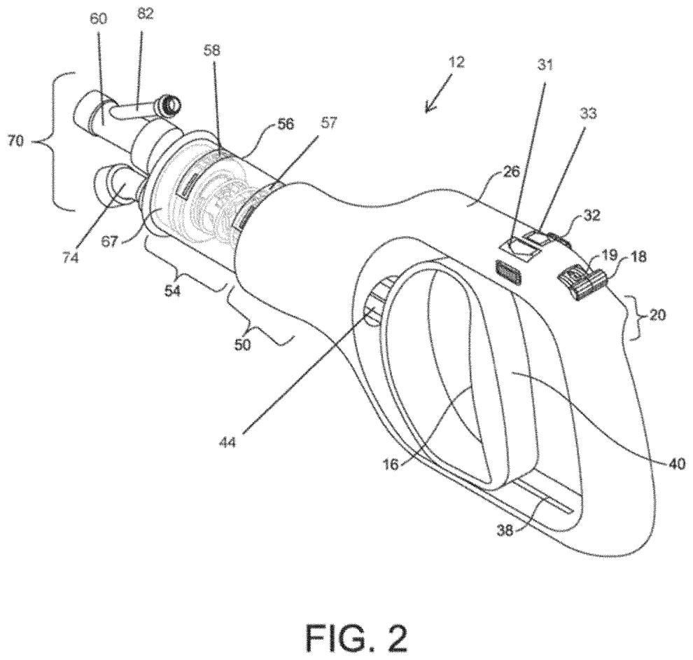

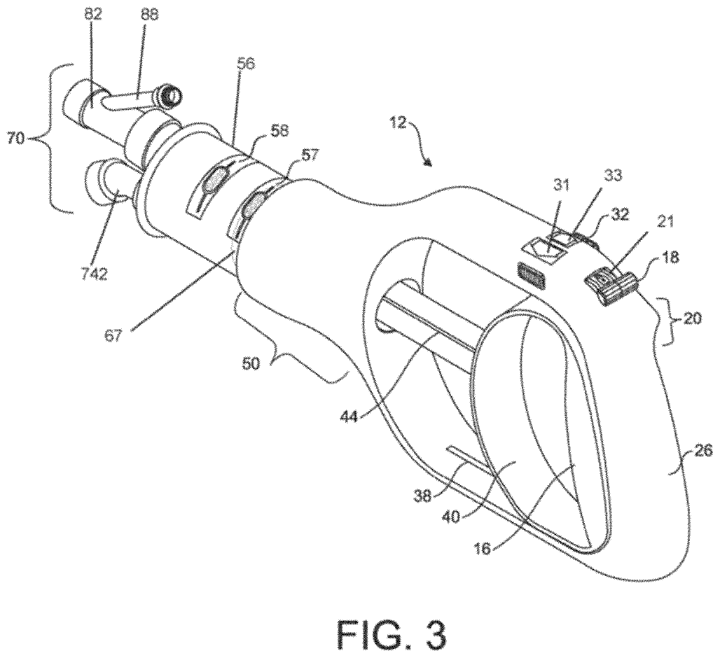

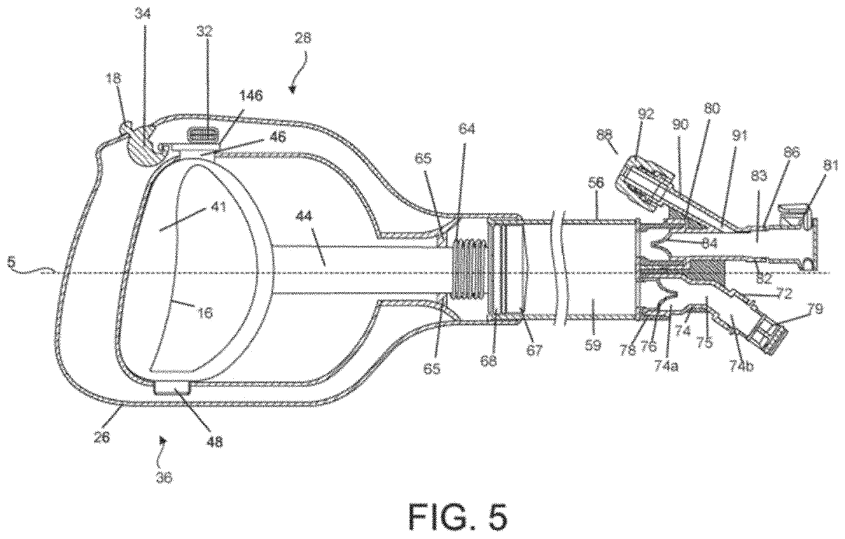

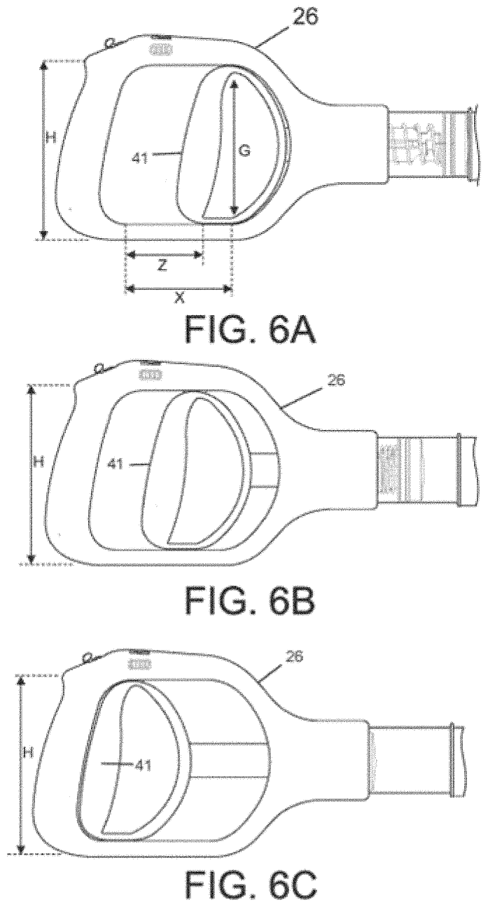

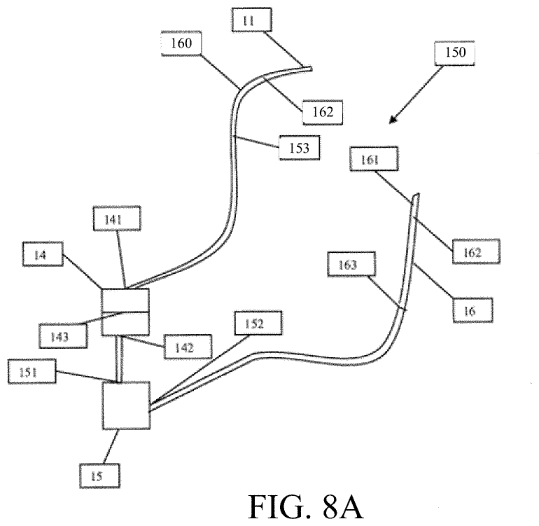

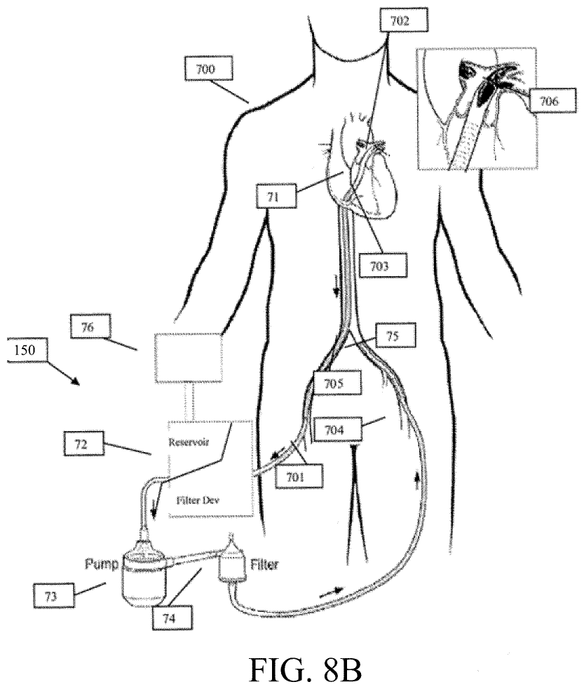

Aspiration systems are designed to facilitate the removal of UIM from a subject. Some aspiration systems can be configured to remove the UIM via a manually controllable handheld device that is configured to aspirate or remove blood or other fluid from the subject and filter the UIM from removed fluid, such as in the embodiments shown in FIGS. 1-7B. Other aspiration systems can be configured to continuously filter and reinfuse the subject's blood to remove the UIM, such as in the embodiments shown in FIGS. 8A and 8B. Each of these embodiments will be generally described below.

Referring to FIGS. 1-7B, there are shown various embodiments of an aspiration system 10 that include a manually operated handheld device. In some embodiments, the aspiration system 10 can be configured to remove a UIM en bloc, particularly using a disposable, manually operated aspiration device coupled to, and in fluid communication with, a suction cannula and waste assembly. The suction cannula comprises an expandable funnel distal end to aid in the en bloc removal of the UIM. The manually operated aspiration device provides for single-handed operation and manual control of generating a suction force and/or a drive force during the removal of UIM from the patient. The aspiration system 100 comprises a suction cannula/procedure sheath subassembly 13, a manually operated aspiration device 12, and a waste collection assembly 112. The aspiration device 12 comprises a handle body 26, a trigger assembly 40, a pump assembly 54, and a connector body 70. The suction cannula/procedural sheath subassembly 13 comprises a suction cannula 94 and a procedural sheath 126. The waste collection assembly 112 comprises a waste collection assembly tubing 118, pinch valves 120, and a waste collection receptacle 114.

The handle subassembly 14 comprises a grip portion 16, vacuum lock actuator 18, a hilt portion 20, a handle body 26, an upper handle slot 30, a lower handle slot 38, a volume limiter, and a vacuum locking mechanism 34. The volume limiter comprises a volume limiter actuator element 32 and a travel stop.

Handle subassembly 14 provides single-handed operator control of fluid aspiration and negative pressure during a clot removal procedure. The handle subassembly 14 is comprised of a handle body 26, trigger assembly 40 and distal handle section 50. Handle body 26 comprises a handle base, an outer gripping surface, an inner gripping surface, handle body upper section 28, handle body lower section 36, and handle distal section 50.

The trigger assembly 40 comprises a resting position (as shown in FIG. 6A), a first activated position (shown in FIG. 6B), and a fully activated position (shown in FIG. 6C). When the trigger assembly 40 is in the resting position, no aspiration or suction force is generated by the pump assembly 54. When the trigger assembly 40 is moved by the user to the active position the aspiration device 12 is configured to generate an aspiration force or a suction force (as described in more detail below). As the user releases their grip on the trigger assembly 40, the trigger assembly 40 is configured to move (as a result of the spring force generated by spring 64) from the activated position to toward the resting position, and the device 12 is configured to generate a drive force (as described in more detail below).

To activate the aspiration, generate vacuum, and/or generate an aspiration force, the user grips the trigger assembly 40, and with their palm resting against the grip portion 16 of handle body 26, pulls the trigger assembly 40 proximally. Handle body 26 and trigger assembly 40 are dimensioned and contoured to facilitate ease of use, optimize user grip and stability during use, and reduce a potential of user hand fatigue during repeated pull cycles. For example, hilt portion 20 of handle body 26 may prevent a user's hand from slipping while gripping or holding the handle body 26; create a contour to securely keep the user's hand in place during use; and/or dimensioned to allow the user to reach the top of device with a finger during use. In one embodiment, handle subassembly 14 is sized to accommodate differences in user hand preferences (left or right hand), hand strengths, and hand sizes. The total length grip portion 16 of handle body 26 (shown as “H” in FIGS. 6A-6C) may have an overall height of approximately 2.5-3.5 inches to accommodate the span of most palm sizes. The maximum trigger span (as shown by “Z” in FIG. 6A) is defined as maximum distance between the trigger assembly 40 at a full rest position (as shown in FIG. 6A) and when trigger assembly 40 is at a maximum activation position (as shown in FIG. 6C). The maximum trigger span is configured to ensure that users, regardless of hand size, can both grasp the trigger assembly 40 and operate the aspiration device 12 within an optimal force range (i.e., move the trigger assembly to any position between the full rest position and the maximum activation position). The maximum trigger span (as shown by “Z” in FIG. 6A) is the total distance in which the lower tab 48 can slideably travel within the lower handle slot 38. In one embodiment, the maximum trigger assembly 40 travel is between 1.2 and 1.7 inches to accommodate the majority of operator hand sizes. The maximum span of the lower handle slot (as shown by “X” in FIG. 6A) is a select distance longer than the maximum trigger span (as shown by “Z” in FIG. 6A).

In one embodiment the handle subassembly 14 is designed as a single use assembly, thereby avoiding the need to re-sterilize the device after each use. However, in another embodiment the handle subassembly 14 may be multi-use and could be re-sterilized or re-purposed between uses if needed.

Trigger assembly 40 provides for single-handed control over aspiration of fluids and undesirable material. When actuated, trigger assembly 40 activates the pump assembly 54 to generate a suction force capable of moving bodily fluids and/or UIM from the vessel, through the suction cannula 94 and into the barrel 56. Trigger assembly 40 is comprised of a trigger handle 41, grasp portion 16, an upper tab 46, and a lower tab 48. Trigger assembly 40 is connected to the plunger rod 44. Grasp portion 16 is configured to be held with the user's fingers. The length of a grip area of grasp portion 16, identified as G on FIG. 6A, is selected to ensure the user can easily actuate the device using some or all fingers. In one embodiment, length G may be approximately 2.5-3.5 inches.

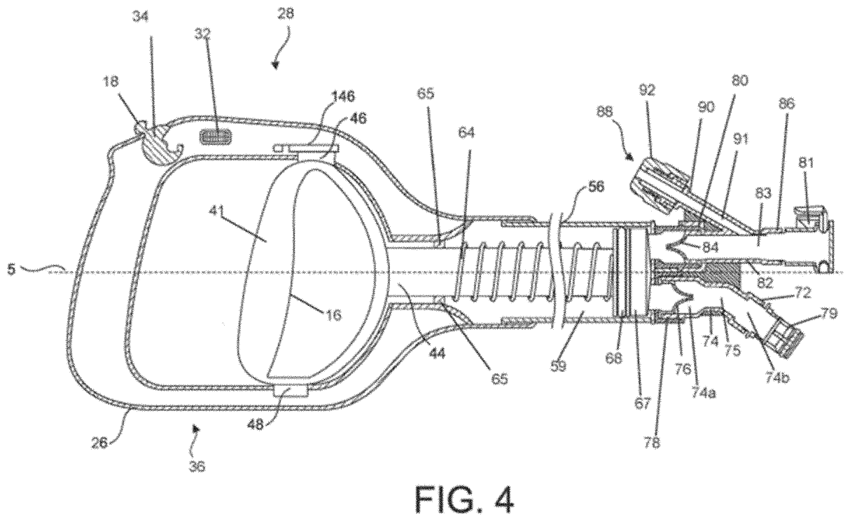

When the trigger assembly 40 is in the resting position (as shown in FIG. 4) the plunger rod 44 is partially disposed within both the barrel cavity 59 and the handle body 26. Spring 64 is coaxially arranged around a portion of the plunger rod 44 and is also partially disposed within both the barrel cavity 59 and the handle body 26. To aspirate fluid and generate a suction force, the user grasps and pulls back (in a proximal direction) on grasp portion 16 of trigger assembly 40. This pulling back motion in a proximal direction generates a suction force as the plunger rod 44, spring 64, and plunger body 67 are longitudinally retracted within barrel 56.

In one embodiment, the volume per trigger pull of aspirated fluid is controlled by a volume limiter element 32, which may be positioned on the handle body upper section 28 (as shown in FIG. 4). The volume limiter comprises a first fluid volume setting 31 and a second fluid volume setting 33. The first fluid volume setting 31 and the second fluid volume setting 33 relate to the amount of bodily fluid to be removed from the patient during a single pull of the trigger assembly 40. In one embodiment, the first fluid volume setting 31 represents a larger volume (e.g., 30 cc) than the second fluid volume setting 33 (e.g., 10 cc). In another embodiment, the first fluid volume setting represents a smaller volume than the second fluid volume setting. The volume settings can be preselected for any volume setting that is equal to or less than the total volume capacity of the barrel cavity 59. The visual indicators 31, 33 comprise any of the following: numbers, letters, shapes, colors, or any other indicia known in the art. In some embodiments, activation and/or deactivation of the volume limiter may include a sound or other tactile feedback.

The volume limiter feature allows a user to select the specific volume to be aspirated in a single trigger pull as well as to change the desired volume per trigger pull at any time during the procedure. The volume limiter is comprised of a volume limiter actuator element 32 and travel stop. Travel stop may be formed as part of the actuator element 32 or as a separate element moveable into the travel path of the trigger 40. When the user engages the volume limiter actuator element 32, travel stop moves into the path of upper tab 46, preventing further proximal travel of trigger assembly 40 thereby limiting the volume of fluid which may be aspirated into barrel 56. In one example, the volume limiter actuator element 32 may have two settings, such as 10 cc and 30 cc. In other non-limiting embodiments, the volume limiter feature may have more settings, such as 10 cc, 20 cc, 30 cc, 40 cc, and/or 50 cc.

As described above, the volume limiter provides the user with the ability to control the amount of blood volume to be removed per trigger pull from the patient during a “search phase” of the procedure and an “active suction phase” of the procedure. The search phase of the procedure is when a user is pulling the trigger 40 to activate aspiration and generate a suction force (thereby removing blood from a patient) prior to the distal end of the suction cannula 94 (for example an expandable funnel 104 distal end) becoming engaged with the UIM. The active suction phase of the procedure is when the user has confirmed that the UIM is engaged with the suction cannula 94 distal end and pulls the trigger 40 to generate active suction force to remove the UIM from the patient's body and into the device.

The reason a user may be required to pull the trigger 40 during a search phase is that the user may not be able to determine if the suction cannula 94 is properly placed and fully engaged with the UIM using only common medical imaging techniques known in the art. If the suction cannula 94 is correctly positioned and engaged with the UIM during the search phase, the user will feel tactile feedback in trigger assembly 40 indicating that the UIM has been engaged by the suction cannula 94. The tactile feedback is generated almost immediately as the vacuum or negative pressure increases, which in turn increases the suction force required to remove the UIM. Furthermore, as the user pulls on the trigger 40 to generate the suction force and engage the UIM, the force exerted on the trigger 40 by the increased negative pressure pulls the trigger 40 distally (i.e., the tactile feedback). This pull on the trigger 40 is therefore felt by the user as tactile feedback indicating to a user that the UIM has been successfully engaged with the suction cannula 94 and the procedure can now transition to the active suction phase. Once the UIM passes through the suction cannula distal end and fluid flow returns, the force required by user to pull the trigger 40 will decrease. In addition to tactile feedback, the user may also receive visual feedback from the system upon engagement with the UIM. For example, the user may visually notice a reduction (or a complete stop) in fluid flow through the system and into the barrel cavity 59 upon the UIM becoming engaged with the system.

Any pulls of the trigger 40 during the search phase will remove a certain amount of volume of blood from the patient, thereby potentially reducing the number of trigger pulls available to the user during the active suction phase (as described in more detail below).

The use of a volume limiter feature solves this problem by reducing the total volume of blood removed per trigger pull during the search phase as compared to the total volume of blood removed per trigger pull during the active suction phase. By way of a non-limited example, the volume limiter may comprise a maximum volume setting of 30 cc and a minimum volume setting of 10 cc. If the volume limiter is engaged to the maximum volume setting then each pull of the trigger 40 will remove 30 cc of blood. If the total volume of blood that can be safely removed from the patient during a single procedure is 600 cc, the user would be limited to a total of twenty trigger pulls at the maximum volume setting (30 cc of blood removed per pull at twenty total pulls equals 600 cc of blood removed). If the user is required to do six trigger pulls during the search phase this would equal 180 cc of blood removed from the patient during the search phase alone; leaving only 420 cc of total blood volume that can be safely removed from the patient for remainder of the procedure. Once the user has received the tactile feedback and/or otherwise confirms the UIM has been engaged with the suction cannula 94 and enters the active suction phase, the user would be limited to a total of fourteen trigger pulls to try and successfully remove the UIM. However, if during the search phase the user engages the minimum volume setting of 10 cc of the volume limiter and during the active suction phase the user then switches the volume setting and engages the maximum volume setting of 30 cc of the volume limiter, the total number of trigger pulls during the active suction phase is increased (as shown below in more detail). For example, if the user is required to do six trigger pulls during the search phase and has the volume limiter set to the minimum volume setting of 10 cc per pull, this would equal 60 cc of blood volume removed from the patient during the search phase alone; leaving 540 cc of total blood volume that is able to be safely removed for remained of the procedure. Once the user has confirmed that the UIM has been engaged with the suction cannula 94 and the procedure transitions to the active suction phase, the user changes the volume limiter actuator element 32 to the maximum volume setting of 30 cc per pull and the user would be limited to at least eighteen trigger pulls to try and successfully remove the UIM. Therefore, in this non-limited example by using the minimum volume setting of the volume limiter during the search phase of the procedure, the user would gain an additional 4 trigger pulls during the active suction phase with the volume limiter actuator element 32 set to the maximum volume setting of 30 cc. These additional trigger pulls during active suction phase likely increases the chances of successfully removing the UIM substantially en bloc. Moreover, the volume limiter feature also provides the user with the ability to switch between the minimum volume setting and the maximum volume setting at any time during the procedure, for example if user needs to “re-enter” the search phase during the procedure (e.g., if the UIM becomes disengaged or an additional UIM in a second treatment site is required to be removed).

The aspiration device 12 can further include a vacuum locking mechanism (not shown) that is designed to be engaged by the user to maintain a constant vacuum (negative pressure) or a continuous suction force within the system without the user having to maintain continuous hand force or continuous pulling (in a proximal direction) on the trigger assembly 40. In one embodiment, the user engages the vacuum lock actuator 18 by applying a first force (i.e., in the proximal direction) upon the vacuum lock actuator 18 with a finger, and a second opposite force (i.e., in the distal direction) upon the vacuum lock actuator 18. The vacuum locking mechanism can further comprise visual indicators to represent the vacuum locking mechanism being engaged or disengaged. The vacuum locking indicators can comprise either a symbol (such as a lock and/or unlock), letters, colors, numbers, or another visual indicator.

During the active suction phase of the procedure, the UIM may become occluded in the distal end of the suction cannula 94. For example, the user may visualize a reduced volume of aspirated blood through the system (for example, little or no visual bodily fluid is seen exiting the suction cannula 94 and/or into the barrel 56) but the user still has tactile feedback from the device 12 that the UIM is still engaged with the suction cannula 94. In this situation the user may activate the vacuum lock mechanism, and this advantageously allows the user to selectively lock the trigger assembly 40 in an active aspiration position, thereby maintaining a constant suction force or vacuum through the suction cannula 94 without requiring the user to physically pull on the trigger assembly 40. The vacuum locking mechanism thereby aids in the usability of the device as the vacuum locking mechanism allows a user to physically release the trigger assembly 40 but continue to maintain constant vacuum and constant section force upon the engaged The continuous vacuum and constant suction force on the UIM may be maintained for the period of time required to remove the UIM successfully en bloc. Using the vacuum locking mechanism saves the user from physically pulling on the trigger assembly 40 for this entire time period, reducing a potential for user hand fatigue during the procedure.

The ancillary port 88 provides access to the treatment site for insertion and removal of ancillary devices such as secondary treatment devices (as described in more detail below), balloon catheters, angiographic catheters, embolic protection devices, wires and the like. Ancillary port 88 may also be used to deliver fluids such as saline, thrombolytic agents, contrast media, and/or other medicine. Additionally, ancillary port 88 may be used to insert a secondary device (as described below in more detail), or a secondary suction cannula (e.g., secondary suction catheter comprising a second expanding funnel and a cannula shaft with a smaller French size than cannula 94) to aid in the removal of the UIM through the ancillary port 88. Ancillary port 88 is comprised of an ancillary port adapter 92, ancillary port lumen 90 extending from the port adapter 92 to the cannula port inflow lumen 83. Ancillary port adapter 92 may be a luer-type fitting with sealing element to prevent the inadvertent introduction of air into the system through the ancillary port lumen 90, a quick connect style fitting, or any other fitting as known in the art.

The location and orientation of ancillary port 88 on aspiration device 12 facilitates ease of use during the procedure. In one embodiment, the ancillary port adaptor 92 faces proximally toward the user, so as to provide easy access by the operator at any time during the procedure, even when gripping the trigger 40 with one hand. In addition, ancillary port lumen 90 is offset from a longitudinal axis 5, shown as angle “B1”, to facilitate introduction to and withdrawal from cannula port lumen. In one nonlimiting aspect, angle B1 is approximately 45-55 degrees and may range from 10 to 70 degrees. In one aspect, ancillary port 88 height as measured from an outer wall of the barrel 56 to the ancillary port adapter 92 opening is approximately 1 inch and range up to 3 inches.

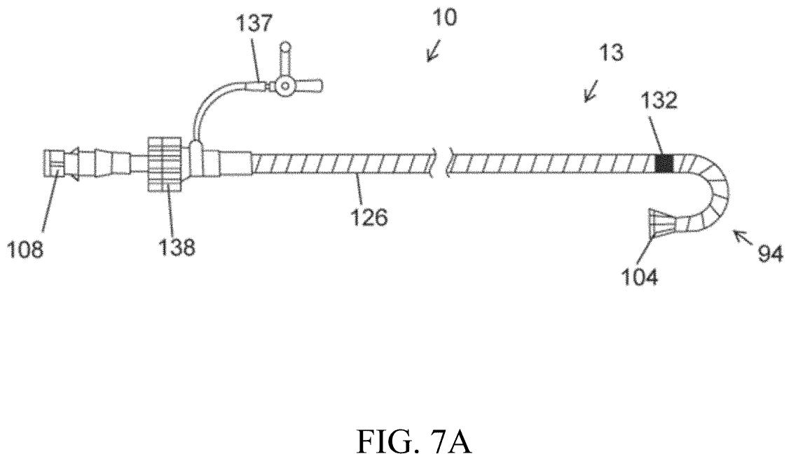

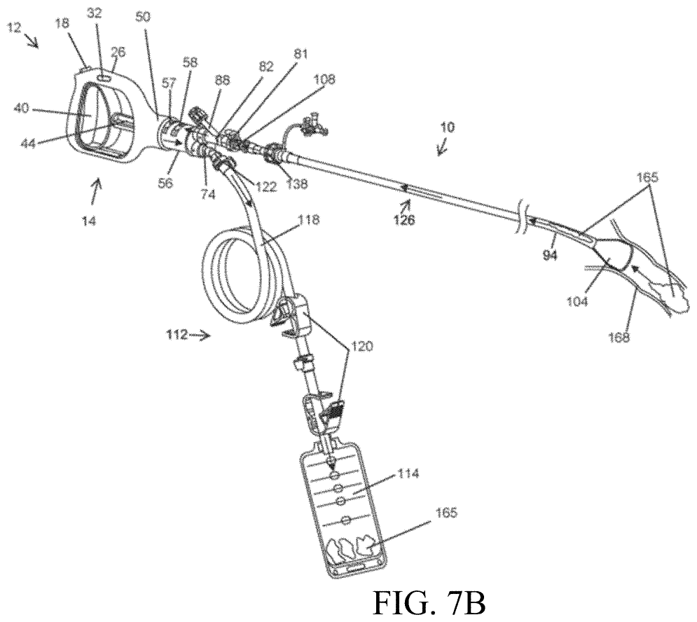

Suction cannula 94 (as shown in FIG. 7) comprises a cannula proximal connector 108, an elongate cannula shaft 96 defining a cannula lumen, and a cannula distal tip section. In one embodiment, the cannula distal tip section comprises an expandable funnel 104. In another embodiment (not shown), the cannula distal tip section comprises a non-expandable member. An aspiration fluid pathway between the treatment site and barrel 56 is established by operatively coupling the cannula's proximal connector 108 to the cannula port connector 81.

Elongate cannula shaft 96 comprises a cannula lumen extending from cannula proximal connector 108 to the cannula distal tip section. In one embodiment (not shown), the cannula shaft may comprise additional lumens, which may extend for a selected distance within or co-axially along cannula shaft, such that the cannula may be a unitary or multi-layer structure. For example, the additional lumens may be used to gain access for a guidewire, secondary device (as described in more detail below), or any other medical device to the treatment site, while simultaneously creating a suction force through the cannula lumen on the UIM. In one embodiment, cannula shaft 96 may be reinforced for enhanced cannula pushability, trackability and/or maneuverability during advancement through the vessel. Such reinforcement may include one or more stiffening elements positioned between and/or around individual shaft layers or embedded within a cannula shaft 96 layer. Reinforcement elements may be in the shape of a coil, weaved material or other patterns. The entire length or selected portions of cannula shaft 96 may be reinforced. In one embodiment, the working length of the cannula shaft 96 may be from approximately 5 cm to 200 cm to accommodate a range of vessel lengths.

The cannula distal tip section of cannula shaft 96 may be pre-shaped to form an angle or curve such that when unconstrained, the expanding funnel 104 becomes offset from the shaft's longitudinal axis (as shown in FIGS. 1, 7, and 8). The offset may be between 10 and 180 degrees. The shaped tip section profile may be formed through standard heat shaping techniques or by utilizing reinforcement elements previously described. The curved tip section is advantageous when engaging a UIM which is partially or fully attached to a vessel wall and when the UIM is located in tortuous or difficult to reach vasculature, such as in a heart chamber or in a pulmonary vasculature.

In one embodiment, the cannula distal tip section comprises an expandable funnel 104 for engaging and moving UIM into a lumen of suction cannula 94. The structural aspects of the funnel 104 including length, profile, structure and flexibility are designed to maximize en bloc clot retrieval while minimizing vessel damage. Funnel 104 has an unexpanded or compressed configuration and an expanded configuration. When in an unexpanded state, funnel 104 may have an outer diameter roughly equivalent to the diameter of cannula shaft 96. In the expanded configuration funnel 104 forms a substantially conical shape with the distal most funnel opening having a diameter larger than the cannula shaft diameter. In one embodiment, the diameter of the funnel opening when fully expanded is approximately 14 mm. The diameter of the funnel 104 may be dictated by the diameter of the target vessel. For example, various sized cannulas 94 comprises varying sized funnel 104 distal ends can be used in combination with the system 12 described here. The wall of funnel 104 may be formed from the cannula shaft 96 or may be comprised of impermeable or semi-impermeable material. The funnel 104 may be self-expanding or mechanically actuated. In one embodiment, the funnel 104 may include a plurality of expandable and independent struts or arms, encased, or otherwise attached to a semipermeable or impermeable membrane layer. Several embodiments of suction cannula 96 are described in more detail in U.S. patent application Ser. No. 16/778,657, published as U.S. Patent Application Pub. No. 2020/0164117 A1, titled SYSTEMS AND METHODS FOR REMOVING UNDESIRABLE MATERIAL WITHIN A CIRCULATORY SYSTEM, filed Jan. 31, 2020, which is incorporated by reference herein in its entirety.

In one embodiment (as shown in FIG. 1 and FIG. 7), the system 10 may comprise a procedural sheath 126 to be operatively coupled to suction cannula 94. When coaxially arranged with suction cannula 94, procedural sheath 126 facilitates insertion and advancement of suction cannula 94. Procedural sheath 126 may also be used to collapse and expand a self-expanding funnel 104 by longitudinal movement of either the sheath 126 and/or cannula 94. Procedural sheath 126 is comprised of a procedural sheath proximal hub 136 comprising a procedural sheath side port 137, and an elongated procedural sheath shaft defining a procedural sheath through lumen which terminates at a procedural sheath end section 132. In one embodiment, procedural sheath proximal hub 136 includes a sealing mechanism which prevents fluid backflow. In another embodiment, procedural sheath proximal hub 136 includes a mechanism 138 to lock and unlock the position of suction cannula 94 relative to the procedural sheath 126. When unlocked, suction cannula 94 may be longitudinally moved relative to the procedural sheath 126 and repositioned relative to the UIM. Procedural sheath proximal hub 136 may be a Tuohy-Borst fitting or other known fittings.

Additional information regarding embodiments of aspirations systems including manually operable handheld devices can be found in U.S. patent application Ser. No. 17/170,782, published as U.S. Patent Application Pub. No. 2021/0275199A1, titled DEVICE AND METHOD FOR MANUAL ASPIRATION AND REMOVAL OF AN UNDESIRABLE MATERIAL, filed Feb. 8, 2021, which is hereby incorporated by reference herein in its entirety.

Referring now to FIGS. 8A and 8B, there is illustrated a system 150 for removing an undesirable material, substantially en bloc, from an obstruction site or site of interest within the vasculature, and for reinfusion of fluid removed (i.e., suctioned or aspirated) from the site of interest back into a patient, in order to minimize fluid loss within the patient. System 150, in an embodiment, may be provided with a first or suction cannula 160 for capturing and removing en bloc the undesirable material from the site of interest, such as that within a blood vessel or a heart chamber. Cannula 160, in an embodiment, may be an elongated tube and may include a distal end 11 through which the undesirable material can be captured and removed. Cannula 160 may also include a lumen or pathway 162 extending along a body portion of cannula 160. Pathway 162, in one embodiment, provides a passage along which the captured material and aspirated circulatory fluid, such as blood, that may be captured therewith may be transported and directed away from the site of interest. Cannula 160 may further include a proximal end 153 in opposing relations to the distal end 11, and through which the captured material may exit from the cannula 160.

Since cannula 160 may be designed for introduction into the vasculature, for instance, through a peripheral blood vessel, and may need to subsequently be maneuvered therealong to the site of interest, cannula 160, in an embodiment, may be made from a pliable material. In addition, as cannula 160 may be used to introduce a suction force to the site of interest for capturing the undesirable material, cannula 160 may be made from a sufficiently stiff material or may be reinforced with a sufficiently stiff material, so as not to collapse under a suction force. In one embodiment, cannula 160 may be constructed from a biocompatible material, such as polyvinyl chloride, polyethylene, polypropylene, polyurethane, polyether block amide (Pebax®), silicone, or a combination thereof.

In certain instances, it may be desirable to maneuver cannula 160 to the site of interest using image guidance, for example, using fluoroscopy or echocardiography. In order to permit cannula 160 to be visualized, cannula 160, in an embodiment, may also include a radioopaque material or any material capable of being visualized.

To better engage and capture the undesirable material substantially en bloc and without significant fragmentation, the distal end 11 of cannula 160 may be designed to have a diameter that can be relatively larger than that of the proximal end 153. In one embodiment, the distal end 11 of cannula 160 may be in the shape of a funnel 20, and may be provided with a diameter, for example, approximately at least three times that of pathway 162. Of course, depending on the surgical procedure being implemented, the ratio between the diameter of funnel 20 and pathway 162 can be varied, if so desired. Funnel 20, with its design, may be placed directly at a site of interest 23 to engage undesirable material 24 or spatially away from the site of interest 23 to capture the undesirable material 24. In a situation where the distal end 11 may be situated spatially away from the site of interest, by providing distal end 11 with funnel 20, a vortex effect may be generated during suctioning to better direct the undesirable material into the funnel 20. It is believed that fluid flowing into funnel 20 can often exhibit a laminar flow circumferentially along the interior surface of the funnel 20 to generate a vortex flow into the distal end 11 of suction cannula 160. Thus, in the presence of a vortex flow, such a flow can act to direct the undesirable material toward the distal end 11 to allow the material to subsequently be pulled into the distal end by suctioning.

To provide a funnel shaped distal end, cannula 160 may include, in an embodiment, a sheath 21 circumferentially situated about distal end 11 of cannula 160. Sheath 21, as illustrated, may be designed to slide toward as well as away from the distal end 11 of cannula 160. In that way, when the distal end 11 is positioned at the site of interest 23, and sheath 21 is retracted (i.e., slid away from the distal end 11), funnel 20 may be exposed and expanded into the desired shape in order to engage undesirable material 24. To collapse funnel 20, sheath 21 may be advanced toward the distal end 11 and over the funnel 20. Thereafter, cannula 160 may be maneuvered from the site of interest 23.

In some instances, the cannula 160 can be deployed downstream of the UIM and advanced towards the UIM for the removal thereof. In other instances, the cannula 160 can be deployed upstream of the undesirable material within a vessel having an arterial circulation (i.e., flow away from the heart), rather than substantially adjacent to the undesirable material. In these implementations, the secondary device could be utilized to substantially occlude the vessel, such that pressure being exerted on the downstream material by the fluid flow can be lessened. By lessening the pressure on the material to be removed, the suction force being applied at the site of interest can act to remove the material more easily.

With reference now to FIG. 8B, there is shown one embodiment of the system of the present invention being utilized for removal of an undesirable material within a patient 700. System 150, as illustrated, includes a suction cannula 71, filter device 72, pump 73, second filter device 74 and reinfusion cannula 75. It should be appreciated that depending on the procedure and to the extent desired, system 150 may not need all of the components shown, or may need other components in addition to those shown.

In general, the method of the present invention, in one embodiment, includes, initially accessing a first blood vessel 701 either by surgical dissection or percutaneously with, for instance, a needle and guide wire. The first blood vessel through which suction cannula 71 may be inserted into patient 700 can be, in an embodiment, any blood vessel that can be accessed percutaneously or by surgical dissection such as femoral vein, femoral artery or jugular vein. Next, suction cannula 71 may be inserted into the first blood vessel 701 over the guide wire, and advanced toward a site of interest 702, for instance, in a second vessel or a heart chamber 703 where an undesirable material 706 may be residing. The second blood vessel or heart chamber, in an embodiment, can be the main pulmonary artery, branch pulmonary arteries, inferior vena cavae, superior vena cavae, deep veins of the pelvic, legs, arms or neck, aorta, or any other medium to large blood vessel for which the use of a cannula is suitable for removing undesirable material without causing undesirable damage to the blood vessel. In addition, the advancement of suction cannula 71 may be gauged or documented by fluoroscopic angiography, echocardiography or other suitable imaging modality.

In the case of pulmonary embolism, the suction cannula 71 may normally be introduced through the femoral, jugular or subclavian vein. Alternatively, the suction cannula 71 may be introduced, if desired, directly into the cardiac chambers using a minimally invasive surgical or endoscopic, thoracoscopic, or pericardioscopic approach.

Thereafter, a third blood vessel 704 may be accessed either by surgical dissection or percutaneously with, for example, a needle and guide wire. Subsequently, reinfusion cannula 75 may be inserted into the third blood vessel 704 using an open or over the guide wire technique. The third blood vessel 704 through which the reinfusion cannula 75 may be inserted, in one embodiment, can be any large vein, such as the femoral vein or jugular vein. Reinfusion cannula 75 may then be advanced toward a reinfusion site, for example, within a fourth blood vessel 705. The fourth blood vessel, in one embodiment, can be the femoral vein, iliac vein, inferior vena cava, superior vena cava or right atrium.

Once reinfusion cannula 75 is in place and components of system 150 have connected, pump 73 may be activated, and suction cannula 71 may then be placed against and in substantial engagement with the undesirable material 706 at the site of interest 702 for removal by suctioning through the suction cannula 71. The undesirable material 706 and circulatory fluid removed from the site of interest 702 may thereafter be directed along suction cannula 71 into filter device 72 where the undesirable material 706 can be entrapped and removed from the fluid flow. The resulting filtered fluid may next be directed downstream by way of pump 73 into the second filter device 74, where any debris or material (e.g., ranging from smaller than microscopic in size to relatively larger) that may have escaped and moved downstream from filter device 74 can be further captured and removed from the fluid flow prior to reinfusion. The resulting cleansed fluid may then be directed into the reinfusion cannula 75 and introduced back into the patient 700.

It should be appreciated that in certain instances, prior to connecting the suction cannula 71 and the reinfusion cannula 75, system 150 may need to be primed with fluid to minimize or eliminate any air and/or air bubbles from the system prior to the initiation of suction and reinfusion. To that end, the suction cannula 71 and reinfusion cannula 75 can be primed separately with fluid or by allowing blood to backfill the cannulae after insertion. The remaining components of the system 150 including all tubing, the filter device 72, the pump 73 and any other components of system 150 may also need to be primed with fluid prior to connecting them to the cannulae. In one embodiment, this can be achieved by temporarily connecting these components in fluid communication with other components as a closed circuit and infusing fluid through a port, while providing another port through which air can be displaced. Once these components have been fully primed with fluid, the circuit can be detached and connected to the primed suction cannula 71 and reinfusion cannula 75 in the appropriate configuration. Examples of a priming fluid include crystalloid, colloid, autologous or heterologous blood, among others.

During operation, pump 73, in one embodiment, may remain activated so that suction and continuous reinfusion of blood can occur continuously for a desired duration or until the removal of the undesirable material has been confirmed, for instance, by visualizing the captured undesirable material in the filter device 72. Alternatively pump 73 can be activated intermittently in short pulses, either automatically or manually by an operator (e.g., surgeon, nurse or any operating room attendant), for a desired duration or until the removal of the undesirable material has been confirmed by visualization of the material within filter device 72.

It should be appreciated that since suction cannula 71 may be deployed within any vessel within patient 700, depending on the procedure, in addition to being placed substantially directly against the undesirable material at the site of interest, suction cannula 71 may be deployed at a location distant from the site of interest where direct engagement with the undesirable material may not be possible or desired.

In a situation where the suction cannula 71 is positioned within a vessel exhibiting a venous flow and at a distant location from the undesirable material, it may be desirable to place the distal end of suction cannula 71 downstream of the undesirable material, so that the fluid flow can push the undesirable material from the site of interest into suction cannula 71 during suction. To the extent there may be some difficulties with suctioning the undesirable material from its location, if necessary, a catheter may be deployed through suction cannula 71 and to the site of interest, where the undesirable material may be dislodged location for subsequent removal.

On the other hand, when suction cannula 71 is positioned within a vessel exhibiting arterial flow and at a distant location from the undesirable material, it may be necessary to place the distal end of suction cannula 71 upstream of the undesirable material for the purposes of removal, even though the undesirable material must move against the fluid flow in order to enter into the suction cannula 71. In such a situation, since the fluid flow in the vessel tends to exert a pressure against the undesirable material at the site of interest, and thus may make the undesirable material difficult to remove, suction cannula 71 may include a flow occlusion mechanism. When expanded radially, the mechanism can substantially occlude the vessel, such that pressure being exerted on the downstream material by the fluid flow can be lessened. By lessening the pressure on the undesirable material to be removed, the suction force being applied at the site of interest can act to remove the material more easily. Again, if necessary, a catheter may be deployed through suction cannula 71 and to the site of interest, where the undesirable material may be dislodged or drawn back into the cannula to facilitate its removal.

Additional information regarding embodiments of aspirations systems including manually operable handheld devices can be found in U.S. patent application Ser. No. 17/170,782, published as U.S. Pat. No. 9,402,938, titled SYSTEM AND METHOD FOR REMOVING UNDESIRABLE MATERIAL WITHIN A CIRCULATORY SYSTEM UTILIZING DURING A SURGICAL PROCEDURE, filed Apr. 11, 2014, which is hereby incorporated by reference herein in its entirety.

In one embodiment, a secondary device, such as a retriever described in detail below, can be used in combination with a suction cannula and an aspiration system (for example the systems described described above) to aid in the removal of the UIM. The secondary device can comprise an elongated body with an expandable element located at a secondary device distal end. The expandable element comprises either an impermeable member, a permeable member, or a member comprising an impermeable portion and a permeable portion. In one embodiment, the secondary device comprises a guidewire member connected to a distal most of end the secondary device. In this embodiment, the guidewire member aids in advancing the secondary device through or to cross a UIM. In one embodiment, the expandable element comprises a self-expanding basket. The basket may be made of a metal material including, but not limited to, stainless steel, nitinol, mesh, high density mesh, compressed high density mesh, wire loops, and/or tether wires. In addition, in some embodiments, one or more regions of the basket may include a coating such as, for example, a silicone or polymer coating. The expandable metal basket comprises a thickness, a pitch, and a length of mesh wires. The thickness, the pitch, and the length of the mesh wires may be designed to control the permeability of the expandable metal basket. For example, in one embodiment the thickness, pitch, and length of the mesh wires of the expandable metal basket are configured such to permit fluid flow through the expandable metal basket distal most end but does not permit UIM to flow therethrough. In another embodiment, the thickness, pitch, and length of the mesh wires of the expandable metal basket are configured such to not permit any fluid flow therethrough, thereby consisting of an impermeable expandable metal basket. Various embodiments of the retrievers are described in greater detail below.

The secondary device is co-axially moveable independently from and within a lumen of the suction cannula. A method of using the secondary cannula of this embodiment comprises co-axially advancing the secondary device distally beyond a distal most end of the suction cannula. In some applications, the secondary device can be utilized to collect the UIM en bloc, i.e., without substantial fragmentation of the UIM. In other applications, the secondary device can be utilized to fragment the UIM and/or clean the interior walls of the anatomical structure. In some applications, the secondary device can then be advanced through or crosses the UIM so that the expandable element of the secondary device is positioned distally beyond the UIM. In other applications, the secondary device can be positioned proximally relative to the UIM. In yet another application, the secondary device can be advanced until the secondary device is positioned within the UIM. Next, a user expands the expandable element distal end of the secondary device. For example, if the expandable element comprises an inflatable balloon the user may inflate the expandable element; or if the expandable element comprises a self-expandable metal basket the user may advance the metal basket out of an introducer sheath. Once the expandable element is activated and in the expanded state, the suction force of the aspiration system may be activated. While the suction force is active the user may retract or pull the secondary device towards and/or co-axially within a lumen of the suction cannula. As the secondary device is retracted or pulled towards the suction cannula the expandable element is configured to engaged with, entrap, mechanically disrupt, and/or macerate the UIM to aid in the removal of the UIM. For example, if the UIM is adhered to an anatomical structure wall the expandable element may mechanically dislodge the UIM from the anatomical structure wall thereby allowing the suction force of the aspiration device to remove the UIM. In another example, if the UIM is occluding the funnel distal end of the suction cannula the expandable element of the secondary device may mechanically squeeze, macerate, and/or force the UIM into the suction cannula lumen for removal. In another embodiment, if the aspiration system is configured to be used in a procedure located in the arterial vascular system, the expandable element of the secondary device can be used as a distal protection device in place of an intravenous filter (as known in the art). For example, in this embodiment the expandable element is designed to be impermeable to the UIM thereby entrapping or blocking any UIM or unwanted debris that becomes dislodged from the treatment site and prevents this material from flowing downstream by fluid flow to the brain or other critical structures in the body to cause additional complications for the patient.

### Retriever Assemblies

Described herein are various embodiments of retrievers (i.e., secondary devices as sometimes described above) for use with aspiration systems and their associated delivery devices, collectively referred to as retriever assemblies. The retriever assemblies are configured to grasp, collect, macerate, dislodge, or otherwise mechanically disrupt the UIM in order to assist in the removal of the UIM from within a vessel or organ (e.g., heart) in a subject's body. UIM tend to be soft or have a sponge-like consistency; therefore, retrievers for use with aspiration systems, such as the aspiration systems 10 described above in connection with FIGS. 1-7B and/or the aspiration systems 150 described above in connection with FIGS. 8A and 8B, need to be specifically designed to have appropriate structures and materials in order to mechanically collect or disrupt the UIM. The retriever assemblies described herein are particularly adapted for the removal of adherent UIM (i.e., UIM that are adhering to the internal walls of the vessel or other anatomical structure) as well. In particular, the retrievers described herein can atraumatically scrape the internal walls of the anatomical structure as the UIM is retrieved, ensuring that the UIM is retrieved en bloc, i.e., without leaving a portion of the UIM mass adhered to the anatomical structure wall and without damaging the anatomical structure wall.

Retrievers or secondary devices for use with the aspiration systems described herein can take a variety of different forms. For example, retrievers could include baskets that are configured to collect or catch the UIM, such as are shown in FIGS. 9-28. As another example, retrievers could include devices that are configured to frictionally engage with the UIM, such as is shown in FIG. 29.

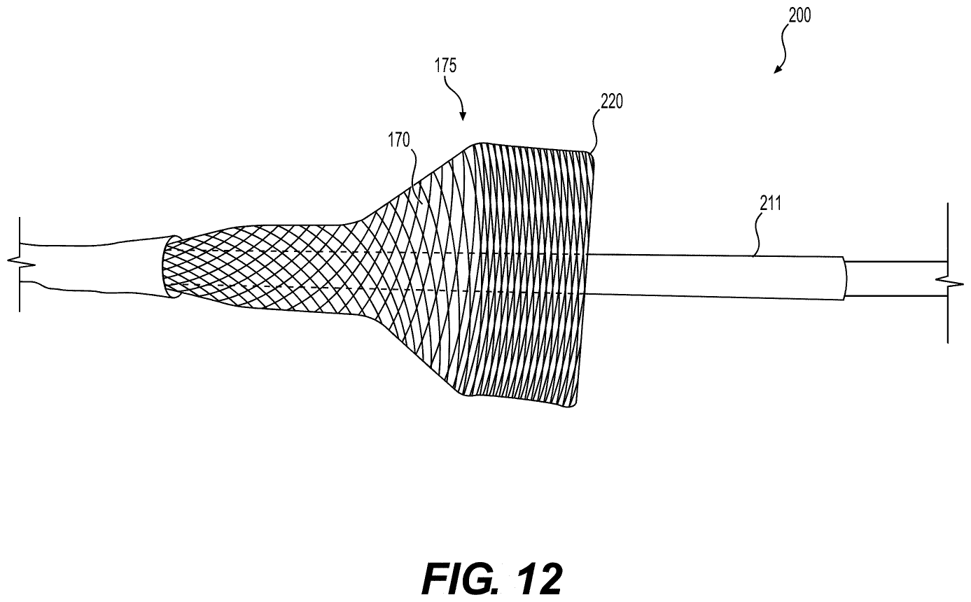

In each of the various embodiments of retrievers described below, the retrievers include a series of struts 170 (FIG. 12) that are interconnected to define a mesh structure 175 (FIG. 12). In some embodiments, the retrievers can be fabricated from flexible materials, including flexible metallic or flexible plastic materials. In some embodiments, the retrievers could be fabricated from a shape memory material, such as nitinol. In some embodiments, the retrievers can be configured to transition between various states or configurations that are particularly adapted for certain purposes, such as a compressed configuration, an expanded configuration, a transitional configuration, a configuration for being delivered to the UIM, a configuration for passing through the UIM, or a configuration for capturing the Throughout the embodiments described herein, the mesh structure 175 of the retrievers and materials from which the retrievers are constructed are advantageous for a number of different reasons. First, these features allow the retrievers to transition between collapsed configurations, which are adapted for being delivered via a catheter through and/or to an anatomical structure, the UIM, a treatment site, or another target structure, and expanded or deployed configurations, which are adapted for engaging and/or collecting the UIM. As described herein, a “collapsed” configuration generally refers to the retriever being radially collapsed to a diameter that is less than the inner diameter of a catheter and/or anatomical structure. The mesh structure of the retrievers facilitates this feature because it allows the retrievers to be collapsed without irreversibly altering the physical structure of the retrievers (i.e., the retrievers can reversibly return to the expanded and/or deployed configurations after being collapsed). Second, the flexibility of the mesh structure and materials allows the retrievers to collect and/or engage the spongy or soft-textured UIM without fragmenting the UIM or unintentionally damaging the inner anatomical structure wall. Therefore, as described in further detail below, some embodiments of the retrievers are adapted for the en bloc removal of UIM from a subject's intravascular system. However, as described in greater detail below, in some applications it could be desirable to fragment or mechanically disrupt the UIM, for example if a distal protection device (such as a filter or aspiration catheter with constant vacuum as described above in FIGS. 8A and 8B) is used in conjunction with the retriever. The retrievers described herein are additionally capable of performing this function.

### Retriever Expandable & Invertible

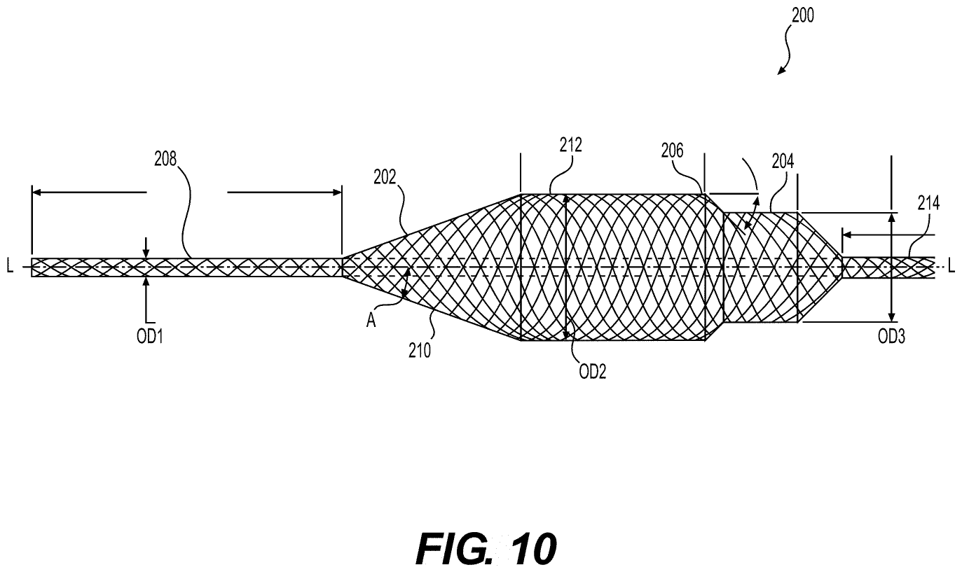

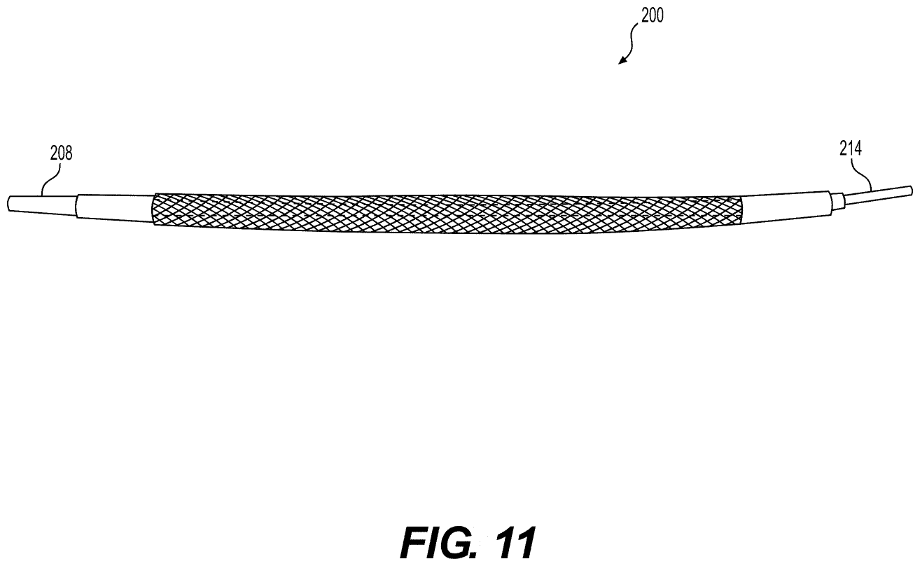

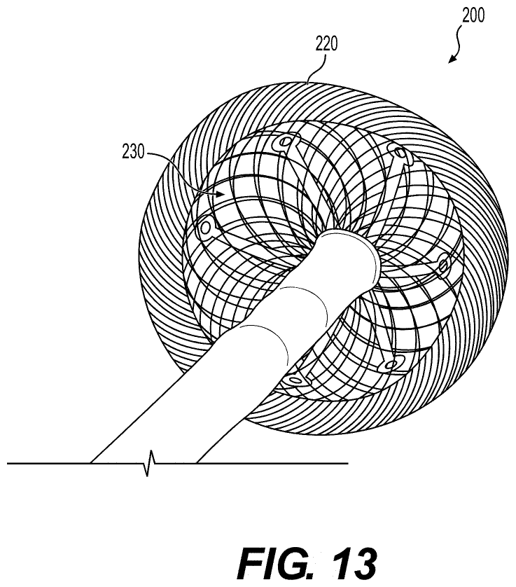

In some embodiments, retrievers for use with the aspiration systems described above could be configured to transition between a first or collapsed configuration, a second or expanded configuration, and a third or deployed configuration. In the collapsed configuration (FIG. 11), the retriever is configured to be delivered through a catheter to the UIM for the removal thereof in a collapsed configuration. In the expanded configuration (FIGS. 9 and 10), the retriever is configured to transition to a diameter that generally conforms to the diameter of the anatomical structure from which the UIM is being removed. In the deployed configuration (FIG. 12), the retriever is configured to collect the UIM in order to draw the UIM towards the distal end of the (e.g., the suction cannula 94 shown in FIGS. 1, 7A, and 7B) for removal from the vessel and/or organ, as described above. As described in more detail below, in some embodiments the retriever transitions from the expanded configuration to the deployed configuration by inverting at least a portion of the retriever. Various embodiments of such retriever assemblies are shown in FIGS. 9-22. In these embodiments, a retriever 200 can include a first section 202 and a second section 204 that are connected at an inversion point 206 (FIG. 10). The second section 204 is configured to invert and collapse into or collapse over the first section 202 at the inversion point 206 to define a basket-shaped structure defining an open interior 230 (FIG. 13) that is sized, shaped, or otherwise configured to receive a UIM therein. In some embodiments, the second section 204 could be configured to collapse in response to the application of a longitudinal force (i.e., a force along the L axis indicated in FIGS. 9 and 10) via a delivery device, as described in greater detail below.

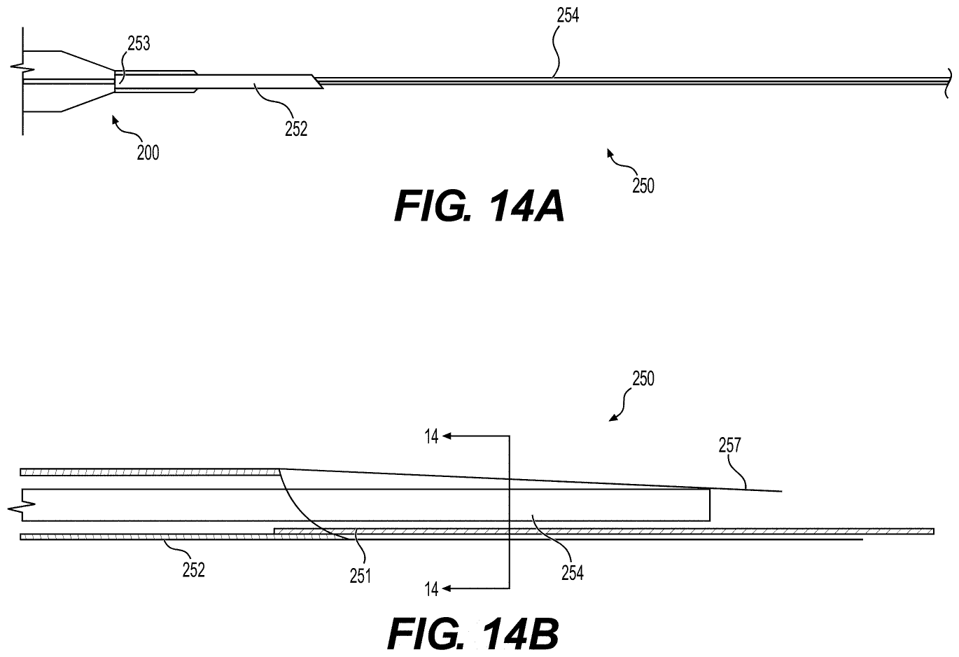

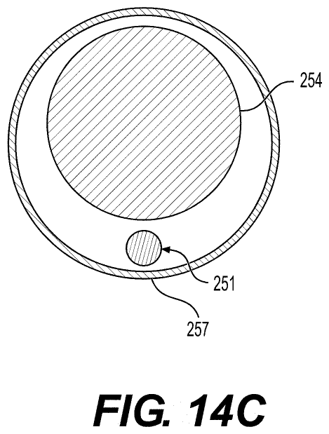

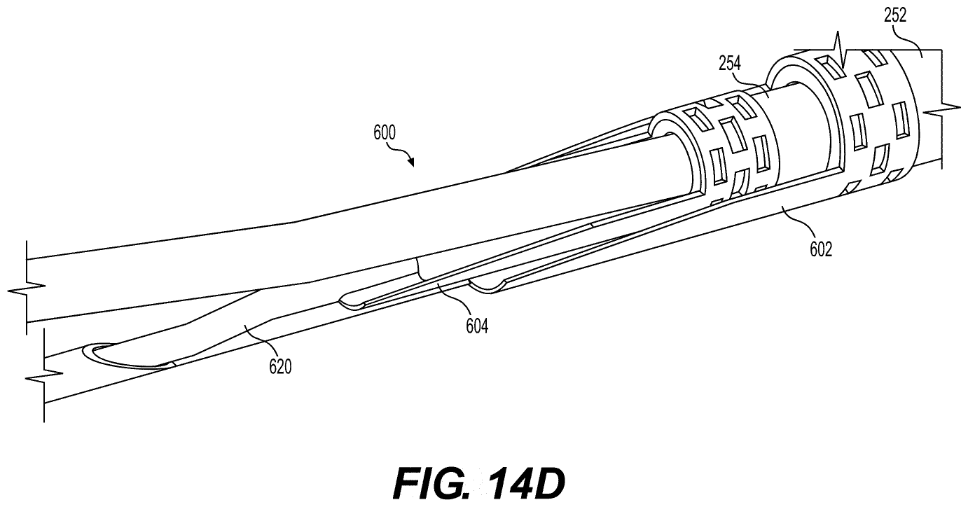

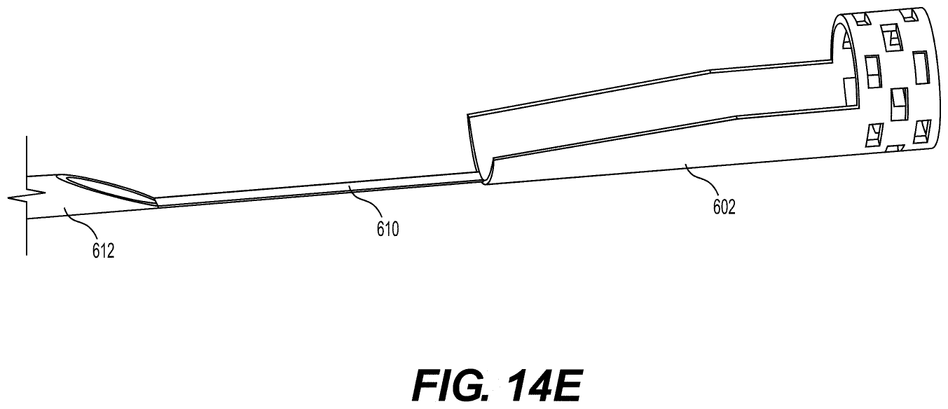

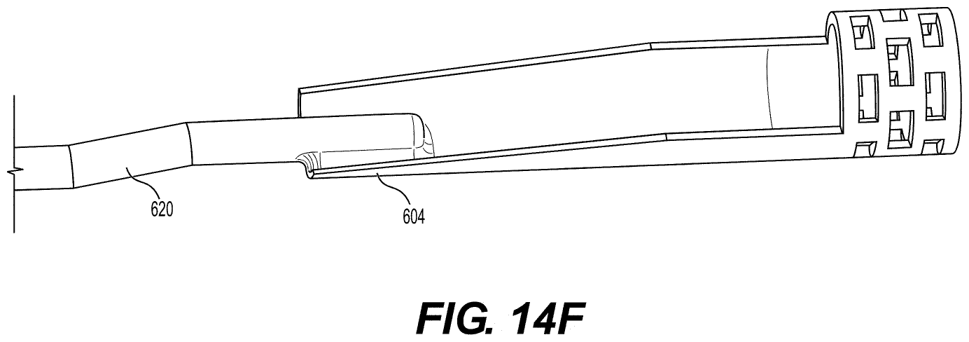

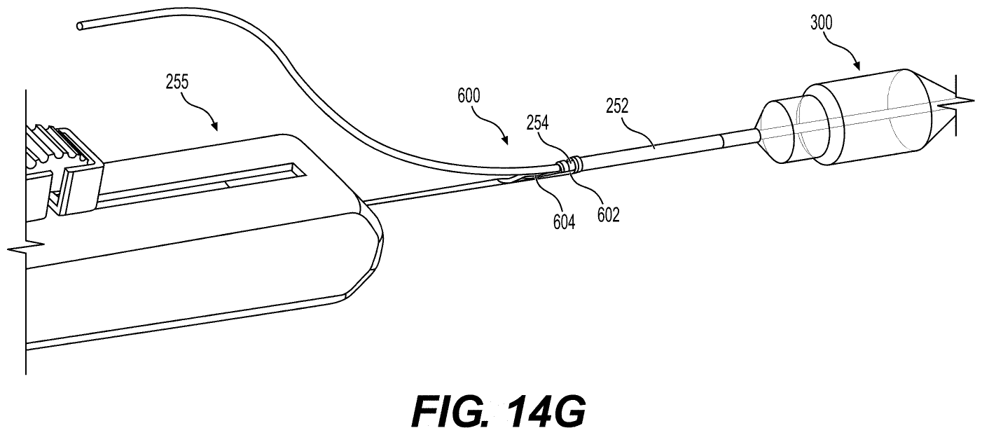

As shown in FIGS. 9-10, the first section 202 can further include a first end 208, a body section 212, and a tapered section 210 connecting the first end 208 and the body section 212. The second section 204 can further include a second end 214. The retriever 200 further includes a shaft 211 that extends through the interior and is slidably movable through the first end 208. In operation, the first end 208 can be coupled to one of the movable portions of the delivery device (e.g., the second shaft 254 of the delivery device 250 in FIGS. 14A and 14B, as described below) and the shaft 211 and/or second end 214 can be coupled to another of the movable portions of the delivery device (e.g., the second shaft 254 of the delivery device 250 in FIGS. 14A and 14B, as described below). In the expanded configuration (FIGS. 9 and 10), the outer diameter of the first end 208 can be less than the outer diameter of the body section 212. In particular, the first end 208 can have a first outer diameter (OD1) and the body section 212 can have a second outer diameter (0D2), wherein the first outer diameter is less than the second outer diameter. In some embodiments, the retriever 200 can be manufactured with varying outer diameters of the body section 212. In particular, the outer diameter of the body section 212 can range from about 8 mm to about 20 mm. As outlined in TABLE 1 below, retrievers 200 having different outer diameters can be suitable for different applications, namely, use in different types of vessels or other anatomical structures. Therefore, the user can preoperatively select a retriever 200 having an outer diameter of the body section 212 that would be appropriate for the type of vessel or other anatomical structure that is being treated. Further, as noted above, the retriever 200 can be flexible (due to the nature of the mesh structure 175 and/or the materials from which it is constructed) and can naturally accommodate variations in the diameter of the anatomical structure in which it is located, without imposing substantial radial forces on the anatomical structure (as long as the retriever 200 is not excessively sized for the anatomical structure). In other words, the retriever 200 can, in some instances, naturally expand to a diameter that corresponds to the internal diameter of the anatomical structure.

TABLE 1

| | | Retriever- | Retriever- | Retriever- |
|---|---|---|---|---|
| | Vessel | 20 mm outer | 12 mm outer | 8 mm outer |
| Vessel | Diameter (mm) | diameter | diameter | diameter |
| Popliteal | 6-10 | Severe | Minor | Optimal |
| | | dilation | dilation | contact |
| Femoral | 10-11 | Moderate | Optimal | Minorly |
| | | dilation | contact | undersized |
| Iliac | 14-16 | Minor | Undersized | Severely |
| | | dilation | | undersized |
| Inferior vena | 18-24 | Optimal | Severely | Severely |
| cava | | contact | undersized | undersized |

In addition to the aforementioned outer diameter range, the various components and/or sections of the retriever 200 can have a variety of different dimensions, some examples of which are set forth below in TABLE 2. In some embodiments, the first section 202 and the second section 204 of the retriever 200 could have the same diameter in the expanded configuration, such as is shown in FIG. 17. In some embodiments, the retriever 200 could include three or more sections, i.e., could include more sections in addition to the first section 202 and the second section 204. For example, FIGS. 18A and 18B show an embodiment of the retriever including a third section 230 positioned between the first section 202 and the second section 204. In this embodiment, the third section 230 includes an additional tapered portion and outer diameter different from the other sections 202, 204.

TABLE 2

| Component | Dimension/Parameter |
|---|---|
| First end (208) length | 10-15 mm |
| Tapered section (210) | 20-30° |
| angle (A, FIG. 10) | |
| Body section (212) | 30-80 mm |
| length | |
| Mesh structure strut | 100-140 μm |
| (170) diameter | |
| Mesh structure strut | 40-50 pics per inch (PPI) |
| (170) density | |

In some embodiments, the retriever 200 can be delivered to the target anatomical structure and transitioned between its configurations via a delivery device. Referring now to FIGS. 14A and 14B, there is shown an embodiment of a delivery device 250 for use in combination with the retrievers 200. The delivery device 250 can be sized, shaped, or otherwise configured such that it can be inserted through the suction cannula of the aspiration system to access the anatomical structure being treated. The delivery device 250 can include a first shaft 252 and a second shaft 254 that are slidably movable with respect to each other. The first shaft 252 includes a lumen through which the second shaft 254 extends. The delivery device 250 further includes a wire or another actuation mechanism 251 that allows the shafts 252, 254 to be moved with respect to each other by a clinician. In the depicted embodiment, the actuation mechanism 251 is coupled to the first shaft 252, such that by pushing or pulling on the actuation mechanism 251, an operator can intraprocedurally slide the first shaft 252 with respect to the second shaft 254. In some embodiments, the delivery device 250 can further include a jacket 257 (FIGS. 14B and 14C) that encloses one or more components of the delivery 250 in order to protect the components from the surgical environment. In the illustrated embodiment, the jacket 257 at least partially encloses the actuation mechanism 251 and the second shaft 254 and the first shaft 252 is slidably movable along the exterior surface of the jacket 257. In other embodiments, the jacket 257 is coupled, adhered, and/or attached to an inner surface of the first shaft 252. In some embodiments, the jacket 257 tapers from the first shaft 252 toward the second shaft 254 and actuation mechanism 251. In this way, the jacket 257, the second shaft 252, and actuation mechanism 251 take up less space within the aspiration cannula, thereby resulting in improved aspiration.

In operation, one of the ends 208, 214 of the retriever 200 is coupled to the first shaft 252 and the other of the ends 208, 214 is coupled to the second shaft 254. Accordingly, by moving one of the shafts 252, 254, while maintaining the other shaft 252, 254 in a fixed position, the delivery device 250 can be utilized to impart a longitudinal force to the retriever 200, which can in turn cause the second section 204 to invert and thereby transition the retriever 200 to the deployed configuration, and in some cases, transition the retriever 200 from the collapsed configuration to the expanded configuration.

Referring back to FIGS. 14A-C, the first shaft 252 is positioned exterior to the second shaft 254 (FIG. 14C) and the actuation mechanism 251 extends along the length of the second shaft 254 between the first and second shafts 252, 254. In some embodiments, the length of the first shaft 252 can be less than the length of the second shaft 254; namely, the first shaft 252 can be truncated proximally (with respect to the aspiration system) as shown in FIGS. 14A and 14B. Since the first shaft 252 has a larger diameter or profile than the second shaft 254, having the second shaft 254 be smaller in diameter than the first shaft 252 and proximally truncating the length of the first shaft 252 can be beneficial because it in turn reduces the overall profile of the delivery device 250, which in turn increases the amount of free proximal lumen space within the suction cannula (which makes it easier to manipulate and operate the delivery device 250 within the suction cannula of the aspiration system) and allows for increased aspiration.

Referring now to FIGS. 14D-F, there are shown various views of an alternative embodiment of a delivery device 250. In this embodiment, the delivery device 250 presents a slimmer profile that provides even further free proximal lumen space for the manipulation and operation of the delivery device 250 within the suction cannula of the aspiration system. In this embodiment, the delivery device 2500 includes a guide assembly 600, which in turn includes a first guide bracket 602 extending from the first shaft 252 and a second guide bracket 604 extending from the second shaft 254. The first and second guide brackets 602, 604 correspond in size and/or shape such that the second guide bracket 604 can be received by and nest within the first guide bracket 602. The first guide bracket 602, shown separately in FIG. 14E, includes a wire portion 610 (e.g., a flat wire) that extends proximally therefrom and connects to a tube 612. The second guide bracket 604, shown separately in FIG. 14F, includes a wire 620 that is sized, shaped, or otherwise configured to be slidably received within the tube 612 of the first guide bracket 612 and mechanically coupled to the handle assembly 255. In operation, guide brackets 602, 604 can be nested together within the cannula or catheter for delivering the retriever 200 and the wire 620 can be coupled to the handle assembly 255. Accordingly, the guide brackets 602, 604 can be slidably actuated with respect to each other, which in turn slidably actuates shafts 252, 254, which in turn can be utilized to transition the retriever 200 between its configurations, as described above. In some embodiments, the guide brackets 602, 604 can be sized, shaped, or otherwise configured to rest flush against each other in order to further minimize the amount of space taken up by the delivery system and, accordingly, maximize the amount of free lumen space.

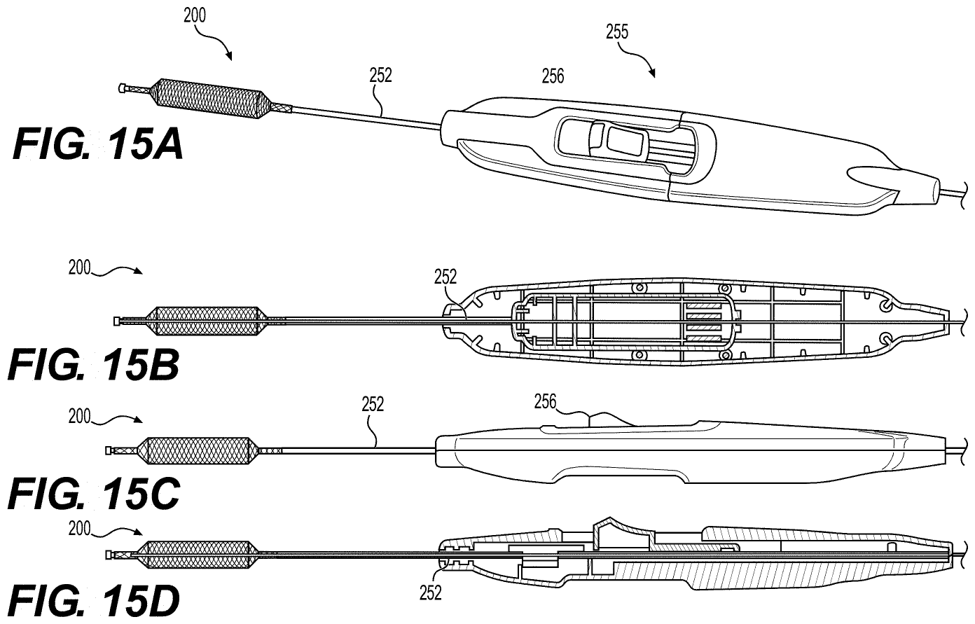

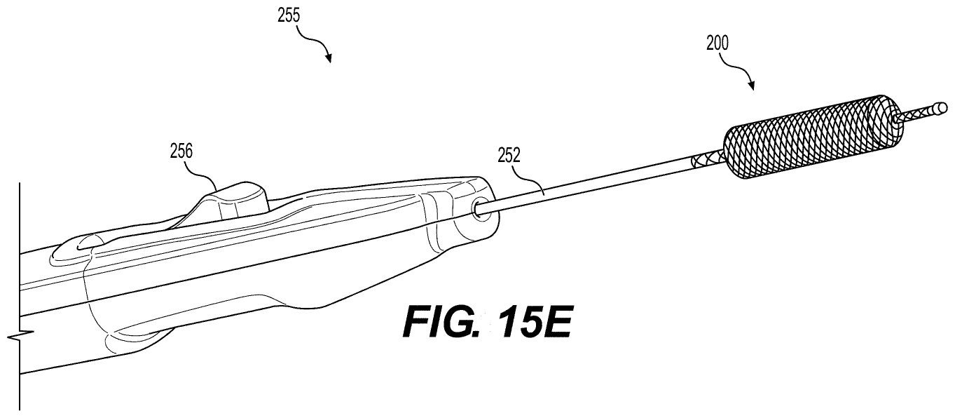

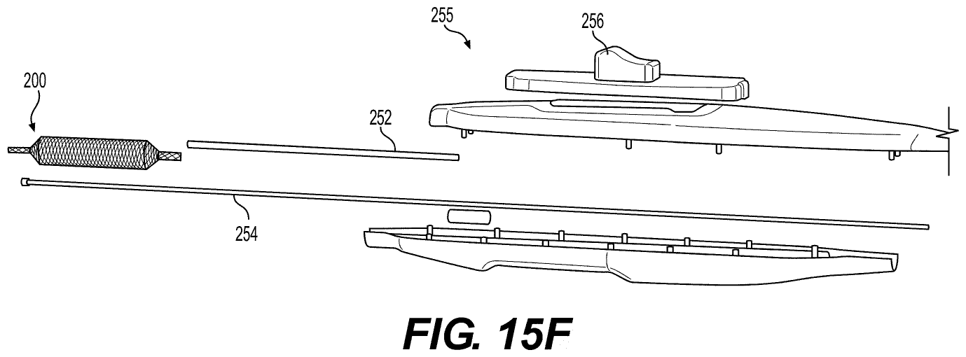

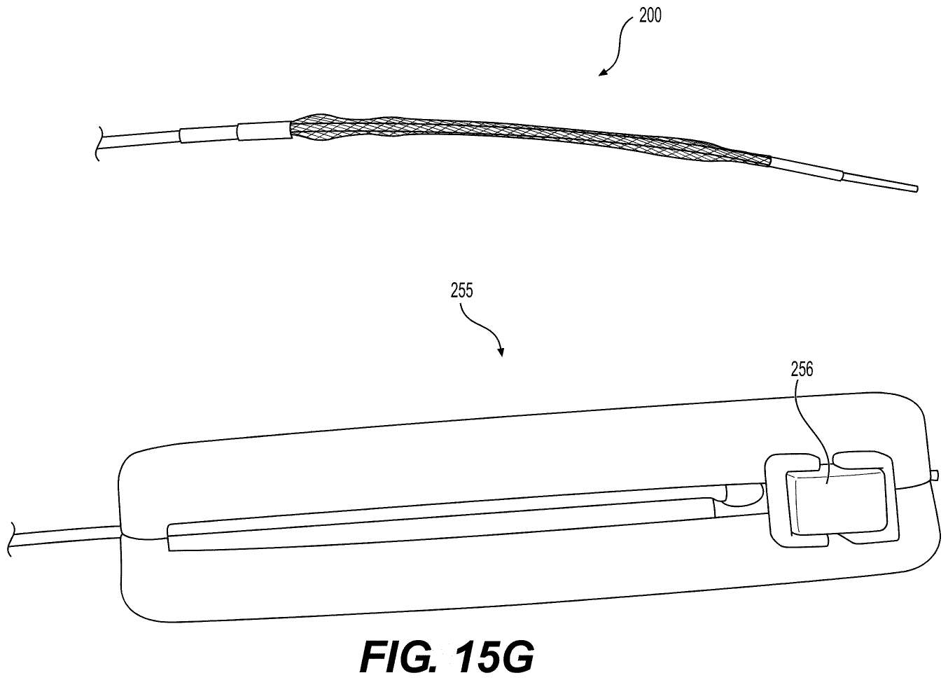

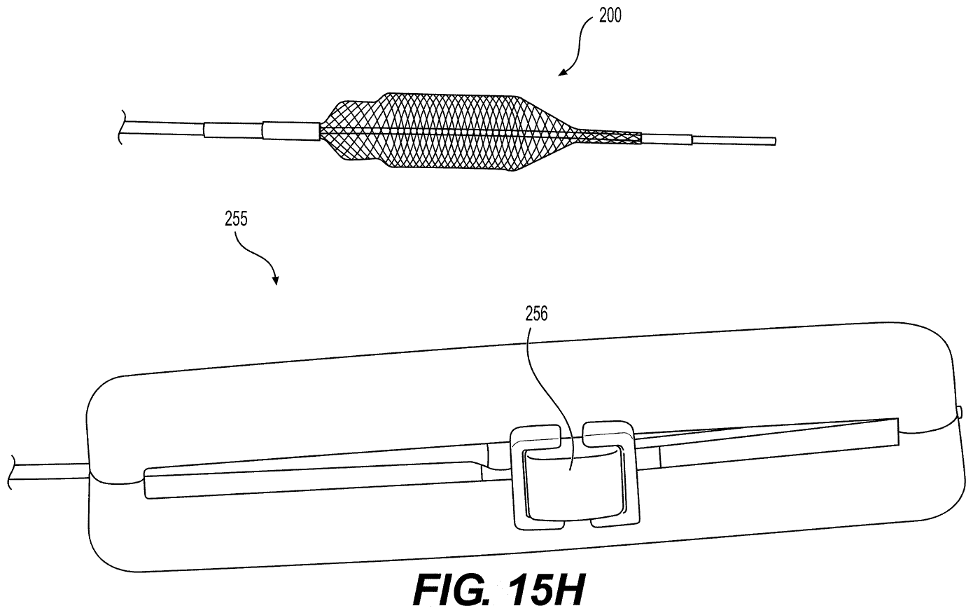

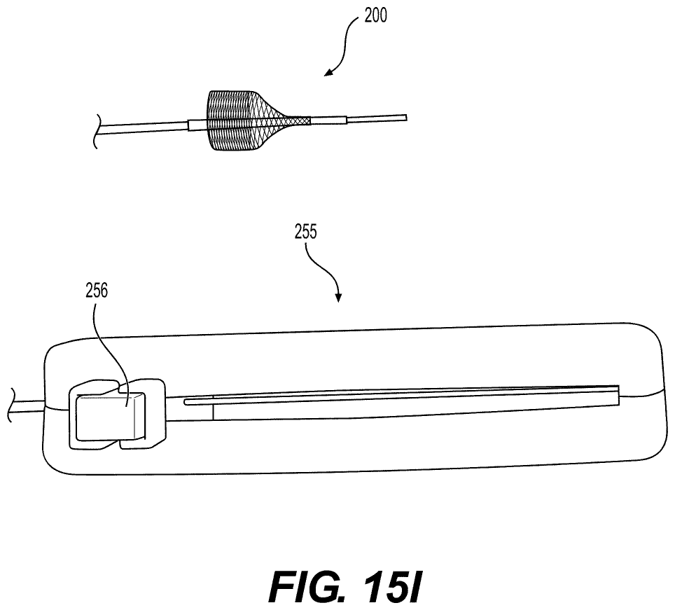

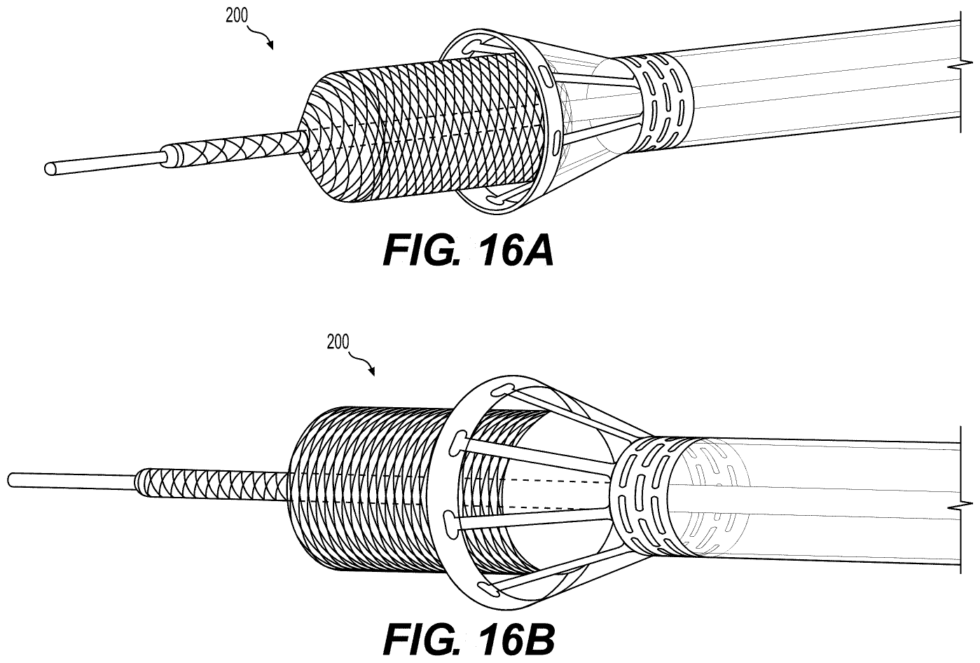

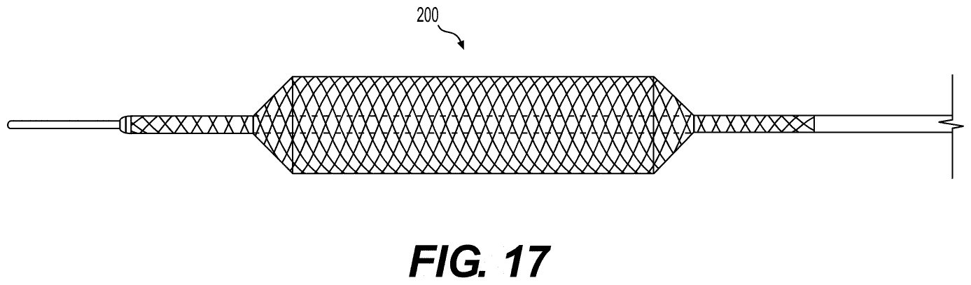

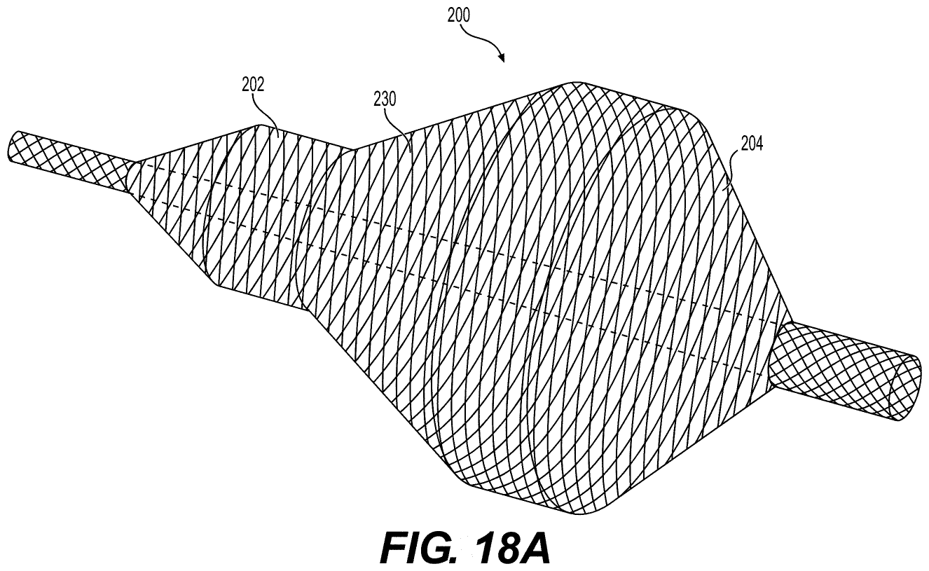

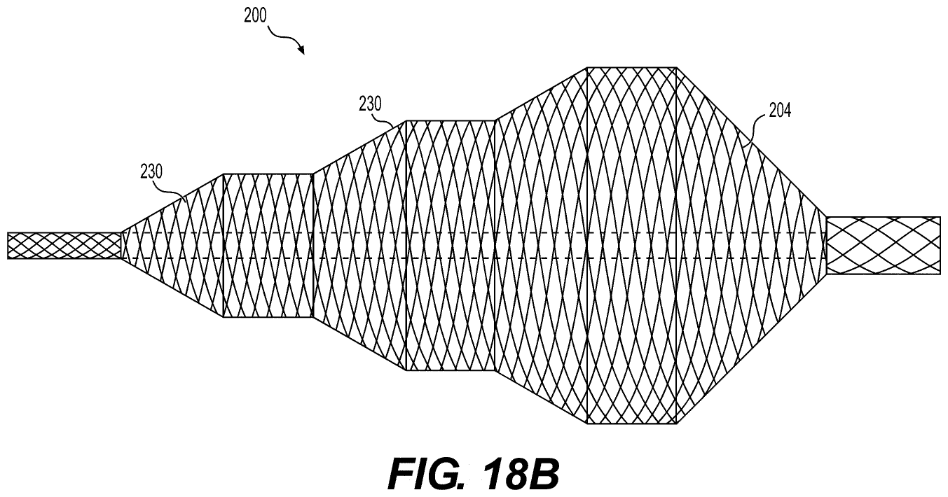

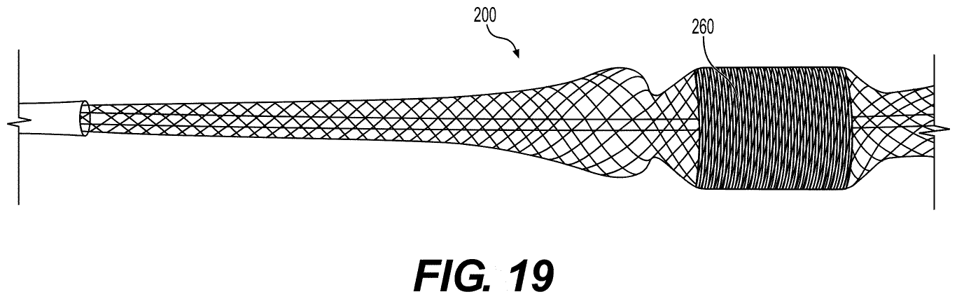

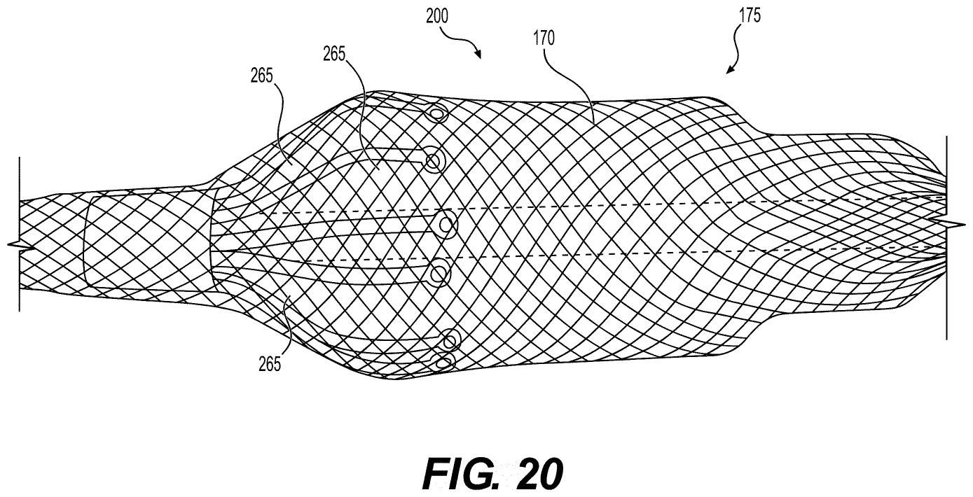

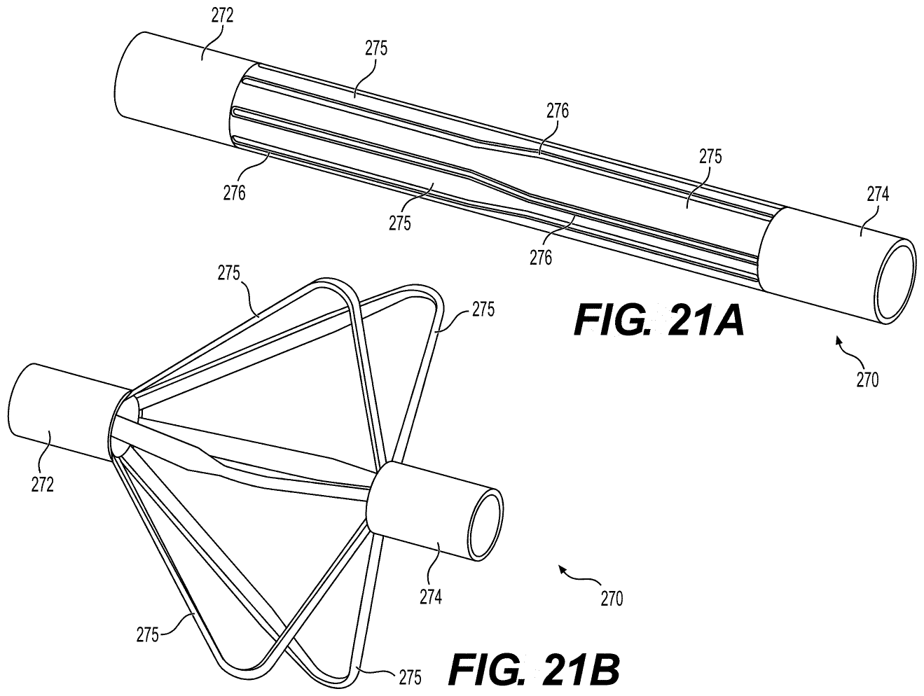

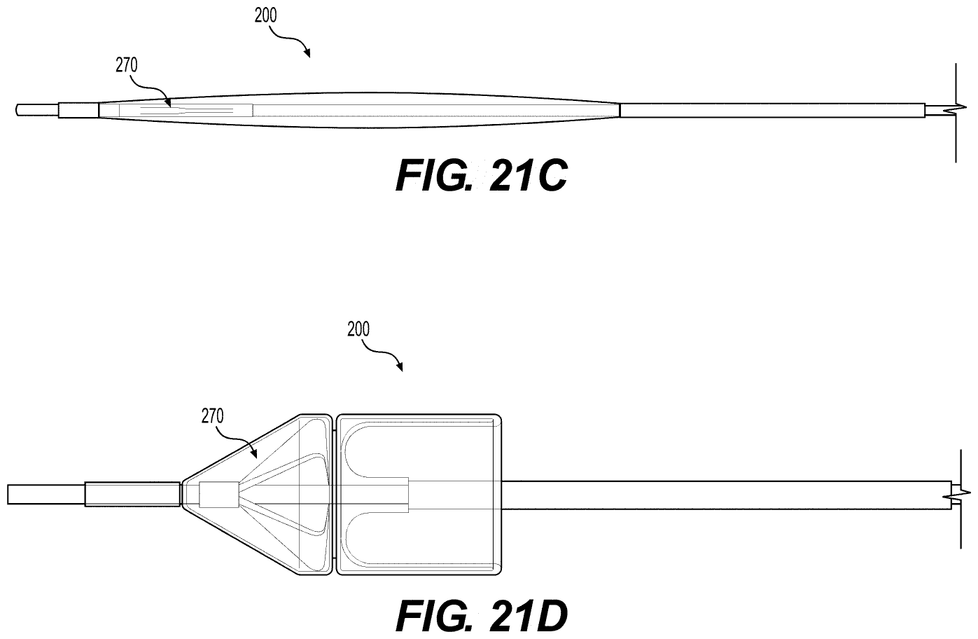

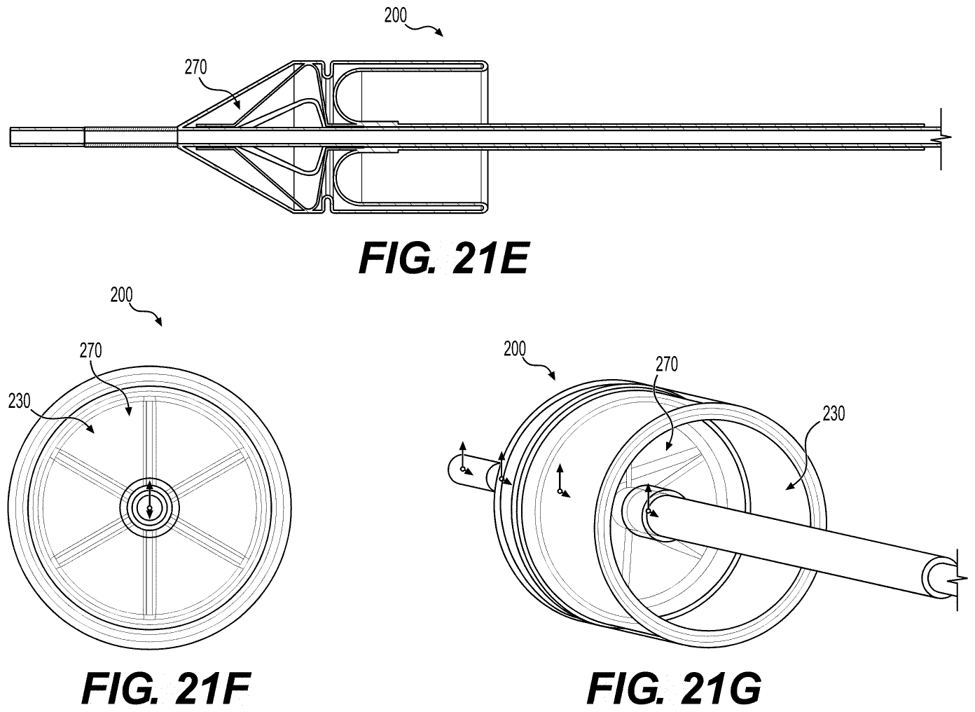

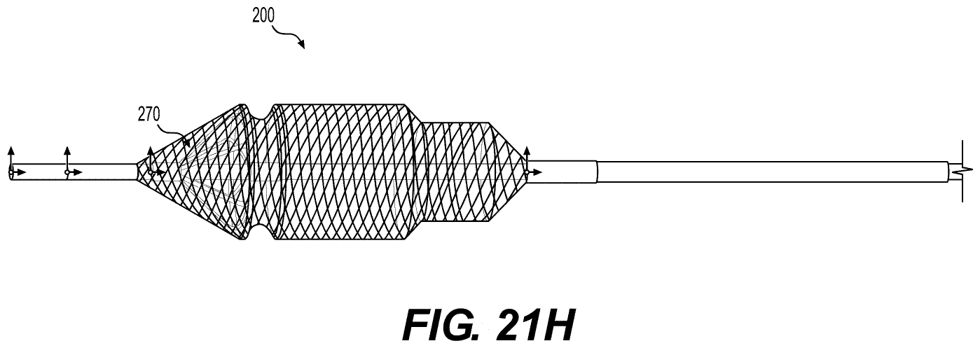

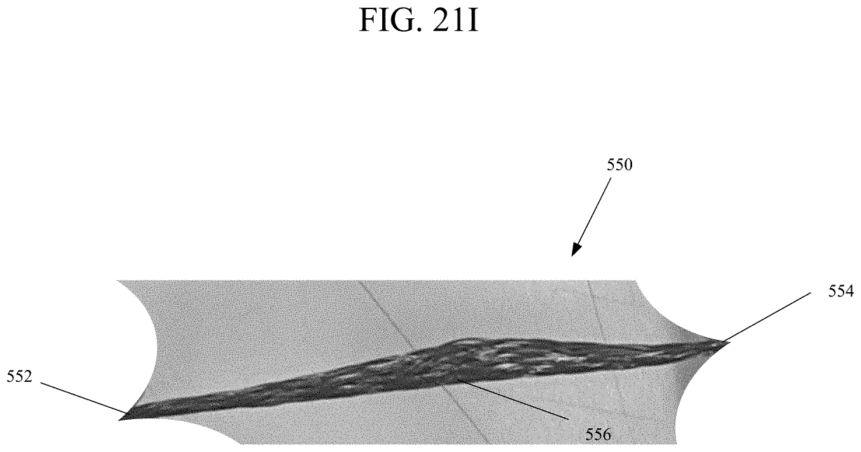

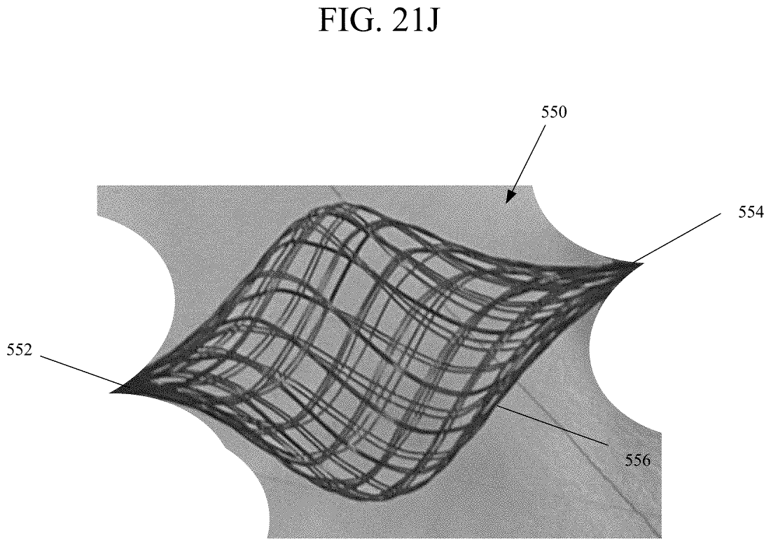

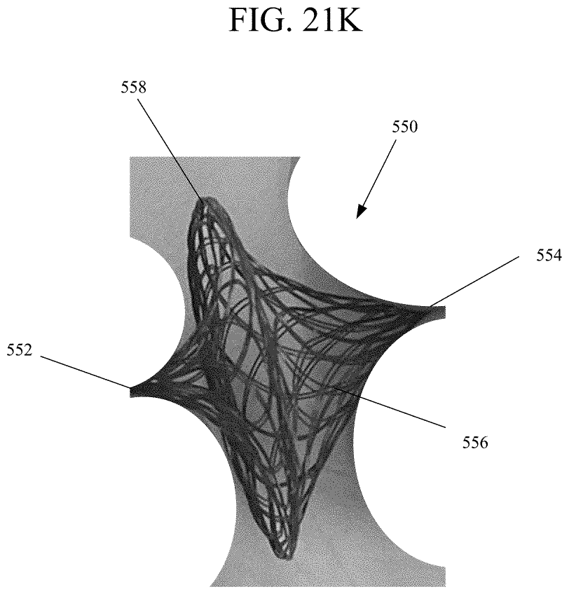

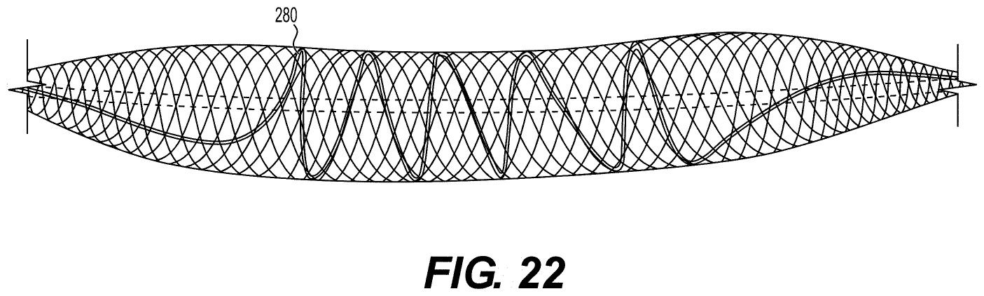

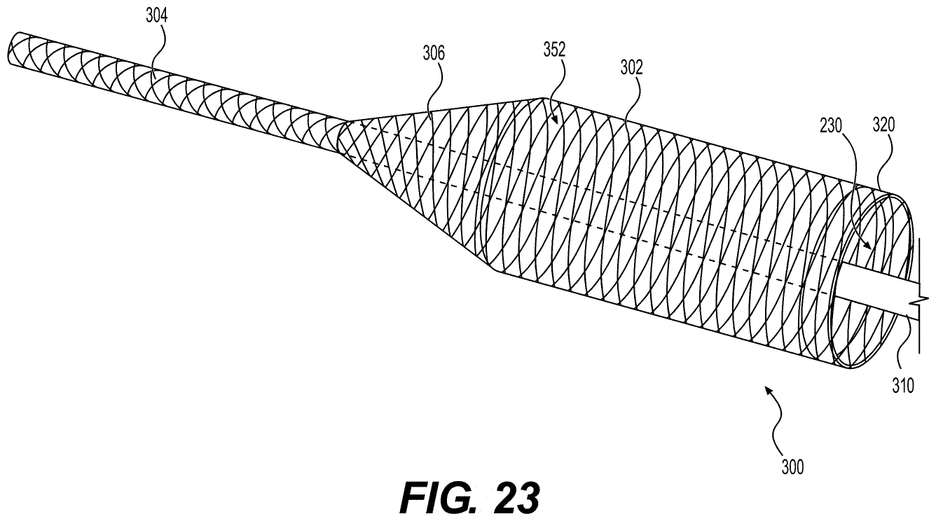

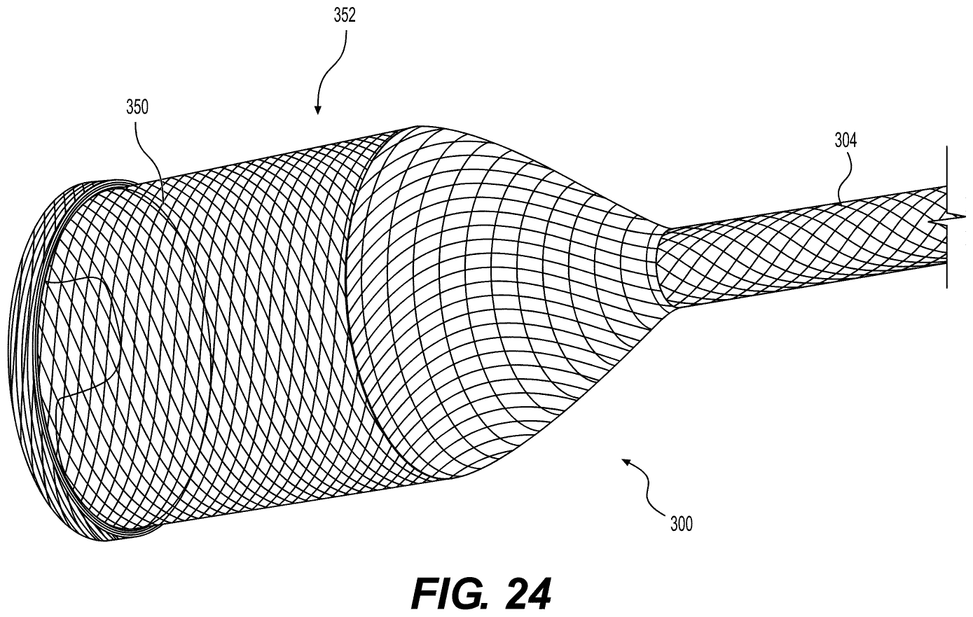

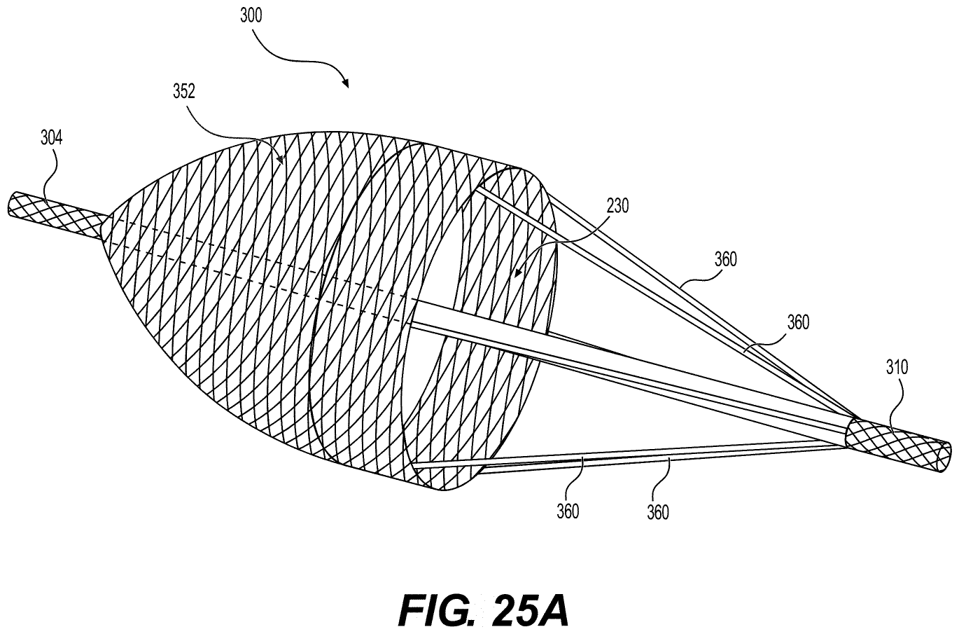

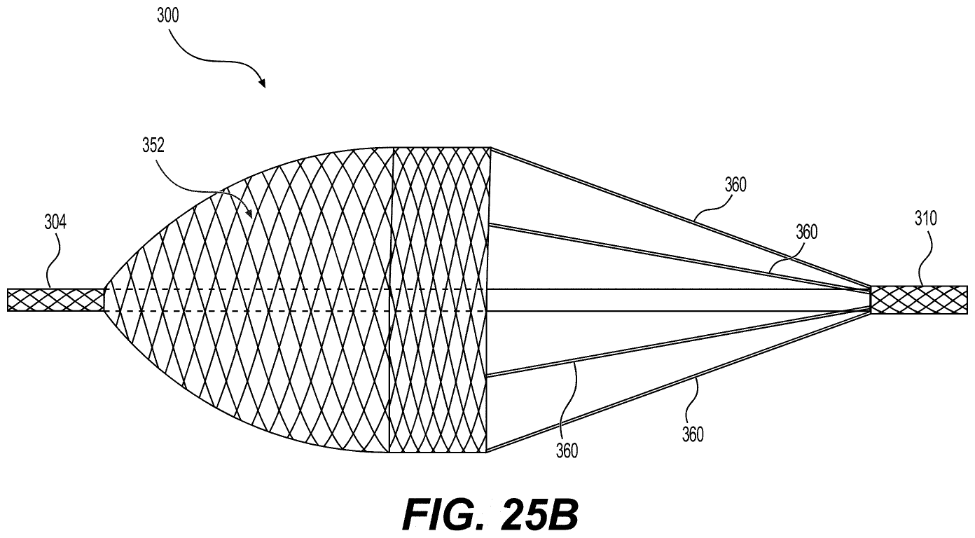

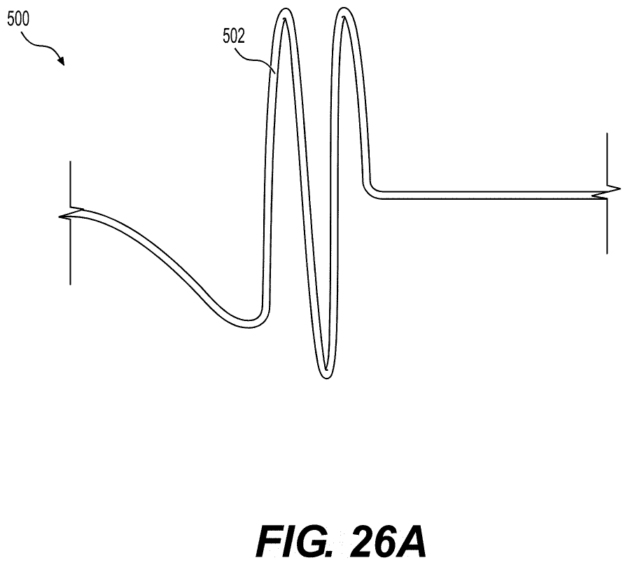

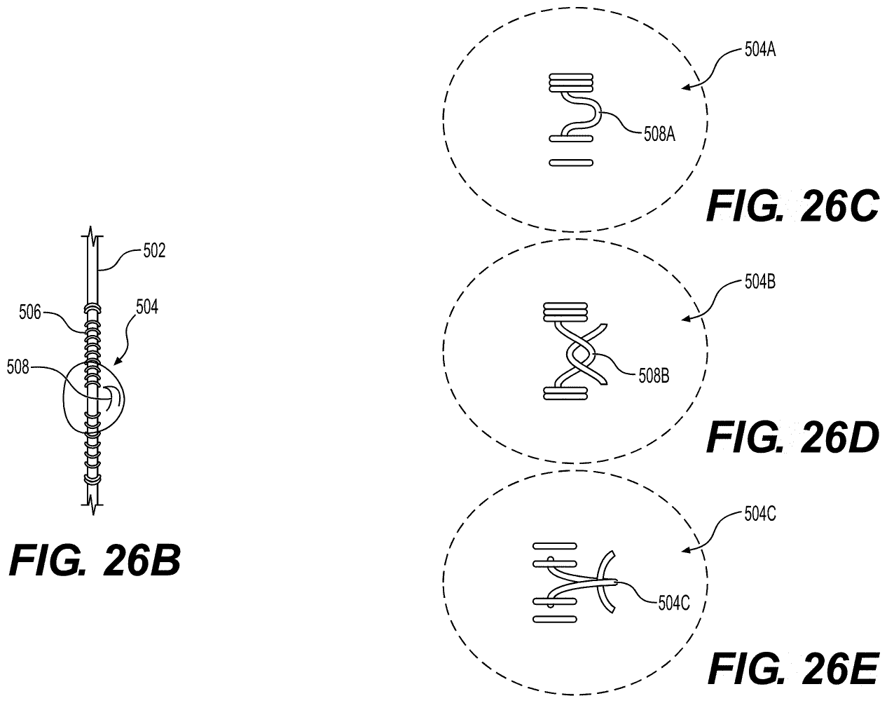

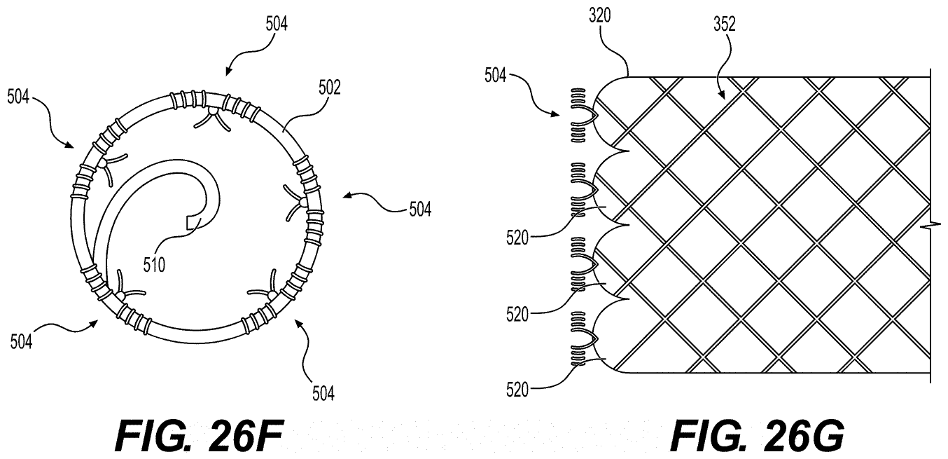

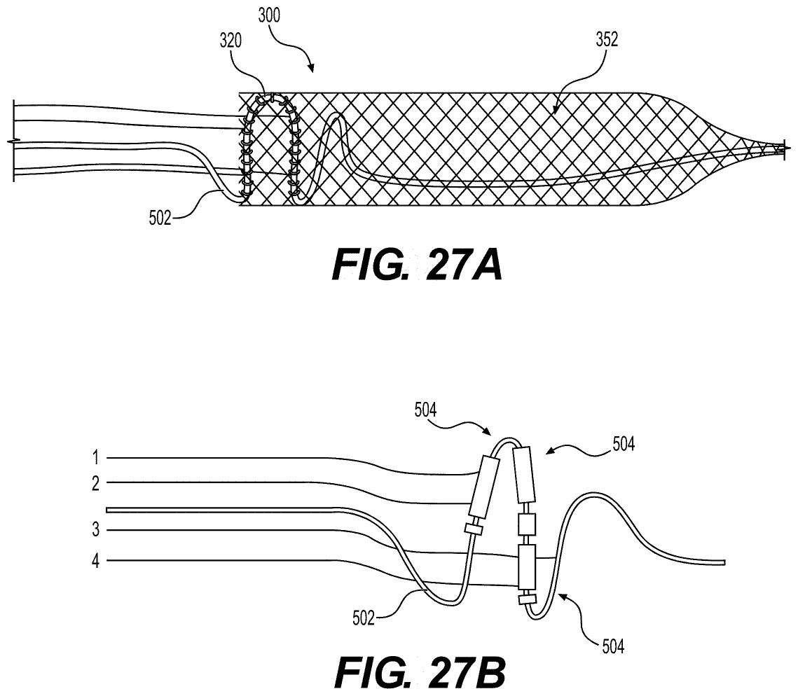

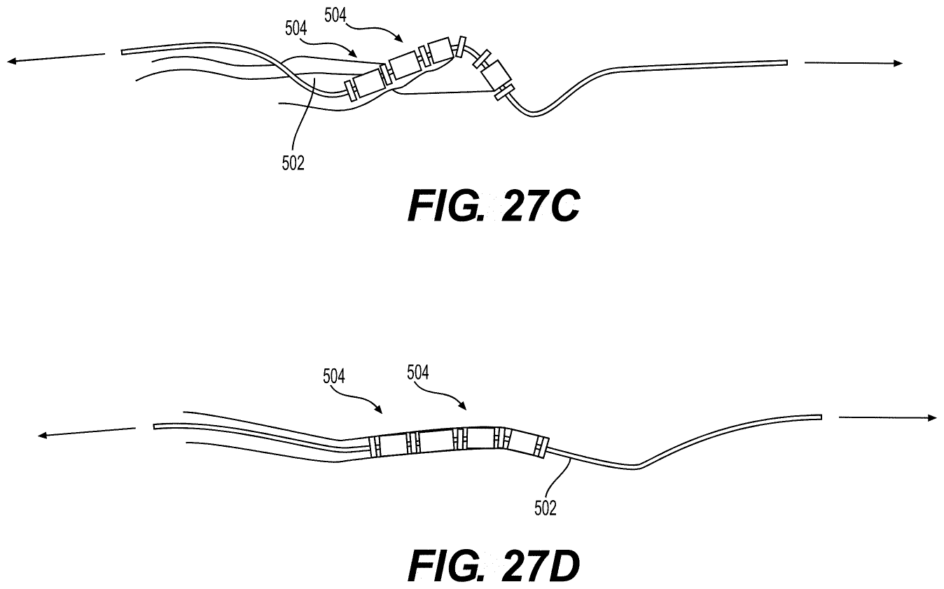

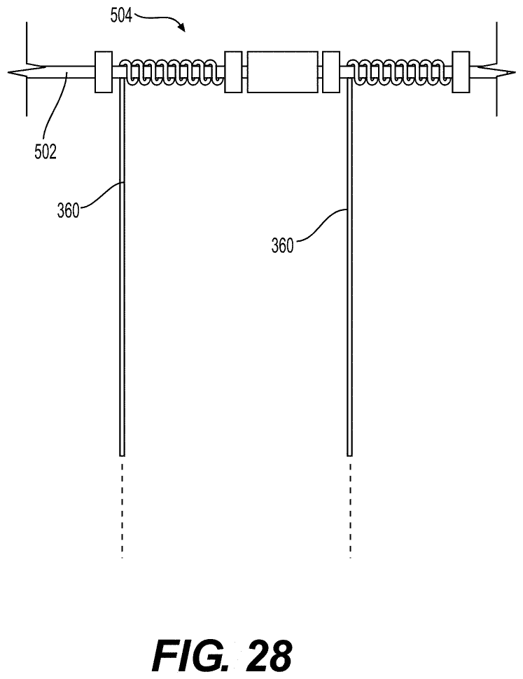

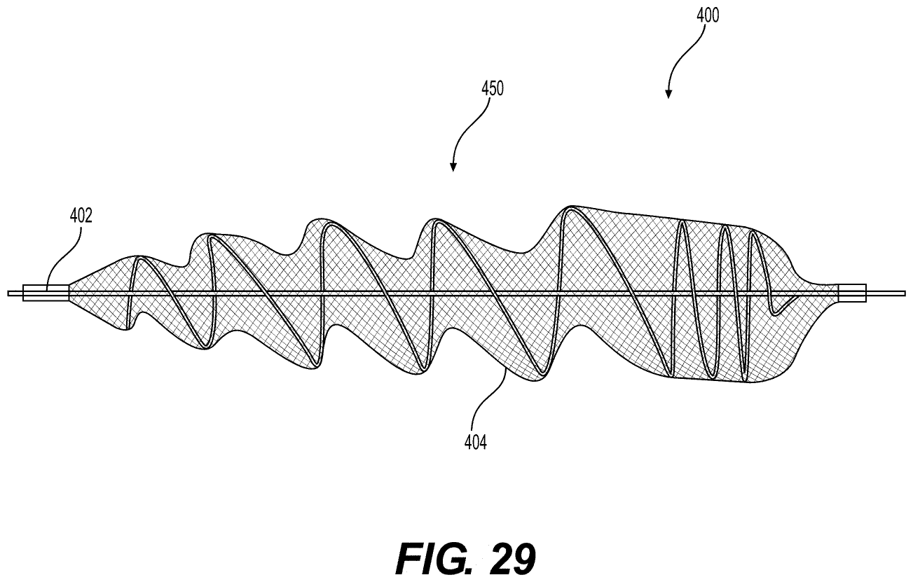

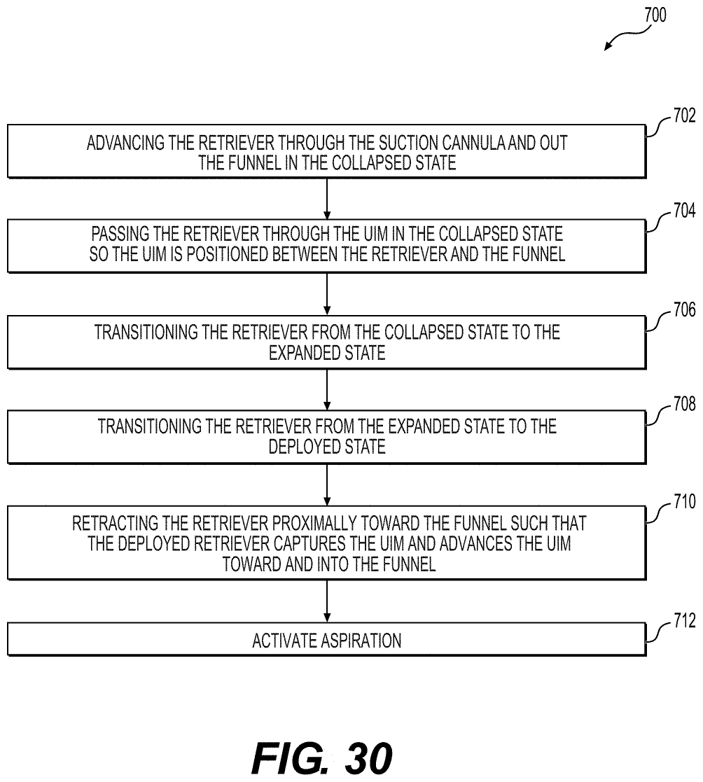

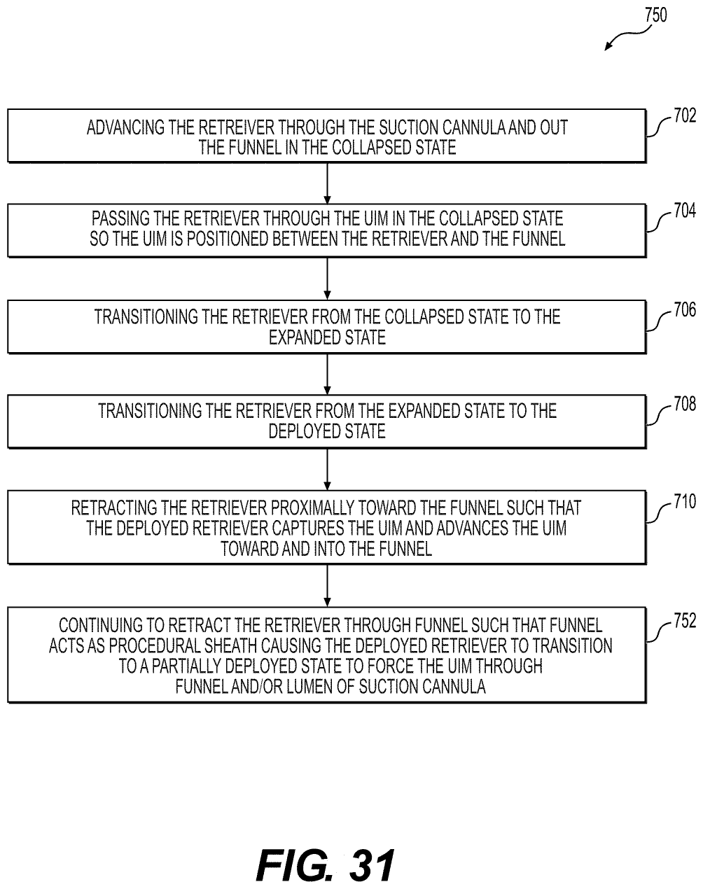

Referring now to FIGS. 15A-I, there are shown various views of a handle assembly 255 for controlling the actuation of the delivery device 250. In the illustrated embodiment, the first shaft 252 of the delivery device 250 can extend through the suction cannula, out of the subject's body and be connected to a handle assembly 255 that is configured to allow an operator to intraprocedurally control the relative positions of one or more of the shafts 252, 254 of the delivery device 250 and, thus, the configuration of the retriever 200. In an alternative embodiment, the actuation mechanism 251 (e.g., a wire) could be coupled to the handle assembly 255 for controlling the relative positions of the shafts 252, 254, as described above in connection with FIGS. 14A-C. In yet another embodiment, the second shaft 254 could be coupled to the handle assembly 255 and controlled thereby. In the illustrated embodiment, the handle assembly 255 includes a control 256 (e.g., a button, slide, or switch) for actuating the first shaft 252 (e.g., directly or via actuation mechanism 251). In particular, the control 256 can be coupled either directly or indirectly (e.g., via an intermediate structure, such as the actuation mechanism 251) to the first shaft 252 such that actuating the control 256 causes the first shaft 252 to translate longitudinally (either proximally or distally) to control the configuration of the retriever 200.

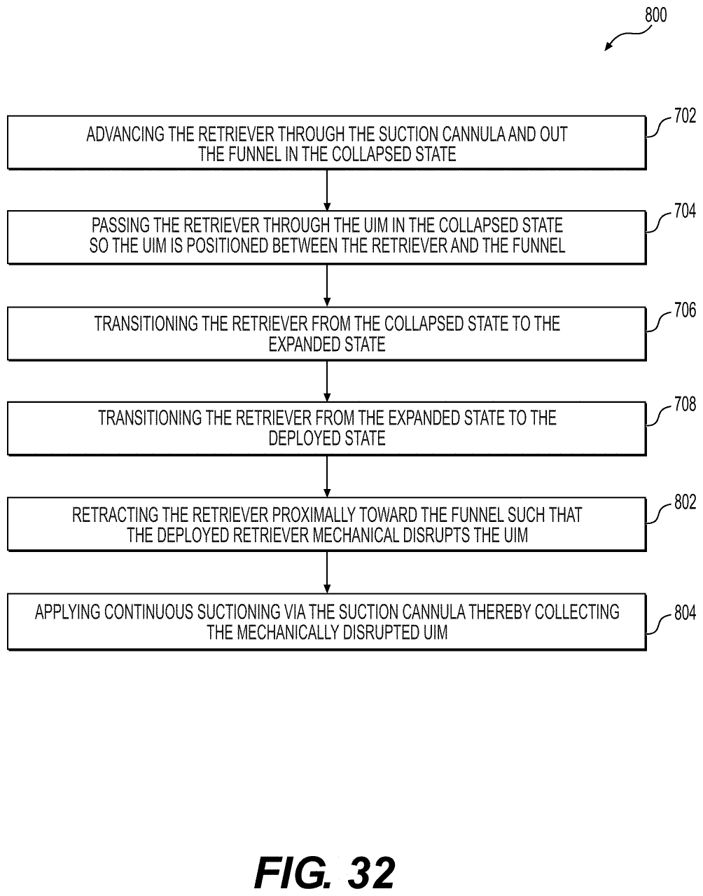

In one embodiment, when the control 256 is in a first position shown in FIG. 15G, the retriever 200 can be in its collapsed configuration. Further, advancing the control 256 to a second position shown in FIG. 15H can cause the actuation mechanism 251 to advance the first shaft 252, which in turn causes the retriever 200 (which is connected thereto) out of the distal end 253 (FIG. 14A) of the delivery device 250 and transition to its expanded configuration within the subject's anatomical structure. Further, advancing the control 256 to a third position shown in FIG. 151, can cause the first shaft 252 to further advance relative to the second shaft 254, which in turn imparts a longitudinal force on the second section 204 (noting that the first section 202 is held in a relatively fixed position since is it connected to the second shaft 254, which is not being advanced by the actual mechanism 251) and causes the second section 204 of the retriever to invert. Accordingly, the handle assembly 255 can be utilized to intraprocedurally actuate the retriever 200 to cause it to transition between its collapsed, expanded, and deployed configurations.

In using aspiration systems (e.g., the aspiration system 10 shown in FIGS. 1-7B or the aspiration system 150 shown in FIGS. 8A and 8B) with the retriever 200, the subject and aspiration system components are prepared as generally described above. After the initial preparation, the targeted anatomical structure (e.g., a vessel 168) is accessed. Accordingly, the retriever 200 is advanced (e.g., via the ancillary port 88 of the aspiration device 12) in the collapsed configuration through a cannula or catheter, such as the suction cannula 94 (FIG. 1) or the suction cannula 160 (FIG. 8A), toward the targeted UIM in the anatomical structure. In some embodiments, the user can monitor the advancement of the retriever 200 under fluoroscopic imaging or via another imaging modality. As the retriever 200 is advanced towards the UIM, it exits the catheter or cannula, expanding from the collapsed configuration (FIG. 11) to the expanded configuration (FIGS. 9 and 10). In various embodiments, the retriever 200 can be configured to automatically unfurl to its expanded configuration or unfurl to the expanded configuration in response to manual actuation by the operator, e.g., by handle assembly 255. Once expanded, a longitudinal force can be applied (e.g., via the delivery device 250) to the second section 204, causing the second section 204 to invert and collapse into or over the first section 202, which in turn causes the retriever 200 to transition to the deployed configuration (FIGS. 12 and 13). Once deployed, the retriever 200 can be advanced towards the UIM, thereby causing the UIM to be mechanically disrupted and/or collected by the deployed retriever 200. In some embodiments, after mechanically disrupting or collecting the UIM, the retriever 200 can be drawn towards the suction cannula to cause the UIM to become engaged therewith (FIGS. 16A and 16B). In some embodiments, as the retriever 200 is retracted, the leading edges 220 (FIGS. 12 and 13) that define the open interior 230 of the basket of the deployed retriever 200 can further be dragged along the interior surface of the target anatomical structure, dislodging the UIM from the walls of the anatomical structure and/or removing any additional or excess UIM therefrom. Further, the aspiration system can be activated to remove the captured and/or mechanically disrupted UIM from the target anatomical structure. Once the UIM has been removed from the subject, the aspiration system and its various components, including the retriever assembly, can accordingly be removed from the subject.

In some embodiments, the retriever 200 can be fabricated from a flexible material, including flexible metallic or flexible plastic materials. In these embodiments, the combination of the flexible material and the mesh structure of the retriever 200 can allow the retriever to collapse into a slim profile, such as is shown in FIG. 11. The ability to collapse into the slim profile can facilitate the delivery of the retriever 200 through the ancillary port 88, the anatomical structure, and/or the UIM during use. In some embodiments, the retriever 200 can be fabricated from a shape-memory material, such as nitinol. In these embodiments, the deployed configuration can be heat set prior to use such that application of the longitudinal force causes the retriever to rapidly transition from the expanded configuration to the deployed configuration.

The inversion point 206 can be formed using a variety of different techniques and/or structures. In one embodiment, the mesh structure 175 is fabricated from a shape-memory material and the deployed configuration can be heat set for the retriever 200 such that the application of the longitudinal force, as described above, causes the retriever 200 to automatically transition to the preset deployed configuration. In another embodiment, the first section 202 and the second section 204 can have different mesh wire densities. In particular, the first section 202 could have a high mesh wire density and the second section 204 could have a low mesh wire density and the inversion point 206 represents the transition between the differing density levels. In use of this embodiment, the application of a longitudinal force to the retriever 200 causes the proximal and distal sections 202, 204 to compress until the higher density first section 202 resists further compression, causing the lower density second section 204 to invert and collapse into the first section 202. In another embodiment, the first section 202 and the second section 204 can have different mesh wire diameters. In particular, the first section 202 could have mesh wire diameter that is larger than the mesh wire diameter of the second section 204 and the inversion point 206 represents the transition between the differing wire diameter sizes. Similarly to the aforementioned embodiment, in use of this embodiment, the application of a longitudinal force to the retriever 200 causes the proximal and distal sections 202, 204 to compress until the higher wire diameter first section 202 resists further compression, causing the lower wire diameter second section 204 to invert and collapse into the first section 202.

In some embodiments, the retriever 200 can further comprise a spring section 260, as shown in FIG. 19. The spring section 260 can be configured to absorb a threshold amount of longitudinal force applied to the second section 204, without causing the second section 204 to invert. Accordingly, an operator of the retriever assembly would have to impart a longitudinal force that exceeds the threshold absorbed by the spring section 260 in order to cause the retriever to transition from the expanded configuration to the deployed configuration. This embodiment can be advantageous because it could prevent the application of minor longitudinal forces from triggering the transition to the deployed configuration, thereby potentially allowing the operators to avoid inadvertently transitioning the retriever 200.

In some embodiments, the retriever 200 can include reinforcement struts 265, as shown in FIG. 20. In the depicted embodiment, the reinforcement struts 265 are positioned internally with respect to the mesh structure 175 of the retriever 200; however, in other embodiments, the reinforcement struts 265 could be positioned externally or formed integrally with the mesh structure 175. The reinforcement struts 265 are configured to radially support the first section 202. The reinforcement struts 265 can be affixed to the internal surface of the mesh structure 175. This embodiment can be advantageous because the radial support provided by the reinforcement struts 265 can prevent the first section 202 from prolapsing intraprocedurally and reinforce the leading edge 220 (FIGS. 12 and 13) of the deployed retriever 200 to improve the ability of the retriever 200 to dislodged an adherent UIM from the corresponding anatomical structure wall. In embodiments where the second section 204 inverts and collapses within the first section 202, the second section 204 could overlay the reinforcement struts 265 within the interior portion 230 of the retriever 200, thereby sandwiching the reinforcement struts 265 between the first section 202 and the second section 204 of the mesh structure 175, as generally shown in FIG. 13.

In some embodiments, the retriever 200 can include a deployable reinforcement strut assembly 270 having reinforcement struts 275 that are configured to extend or deploy in response to the retriever 200 being converted to the deployed configuration, as shown in FIGS. 21A-21H. In the depicted embodiment, deployable reinforcement strut assembly 270 is positioned internally with respect to the mesh structure 175 of the retriever 200. When in the deployed configuration, the reinforcement struts 275 are configured to radially support the first section 202 of the retriever 200 and have the associated advantages generally described above with respect to the embodiment depicted in FIG. 20. In the depicted embodiment, the deployable reinforcement strut assembly 270 includes a first end 272 and a second end 274 that are connected by a series of struts 275 that are delineated via a corresponding series of cutouts 278 extending therebetween. In one embodiment, the first end 272 can be coupled to the distal portion end of the first section 202 and the second end 274 can be coupled to the proximal portion of the second section 204. In another embodiment, one of the ends 272, 274 could be coupled to the corresponding section 202, 204 of the retriever mesh structure 175 and the other end could be unconnected or free floating, i.e., the reinforcement strut assembly 270 could cantilever from one of the sections 202, 204 of the retriever mesh structure 175. In either embodiment, the reinforcement struct assembly 270 is configured to move in conjunction with the movement of the corresponding sections 202, 204 of the retriever 200.

When in the undeployed configuration shown in FIG. 21A, the deployable reinforcement strut assembly 270 is sized, shaped, or otherwise configured such that it can reside within the collapsed interior of the retriever 200. In other words, in the undeployed configuration, the deployable reinforcement strut assembly 270 presents a slim enough profile such that it can reside within the interior volume of the retriever 200 without substantially impacting the profile of the retriever 200. When the retriever 200 is transitioned to the deployed configuration, as shown in FIG. 21D, the movement of the second section 204 towards the first section 202 correspondingly causes the second end 274 of the reinforcement strut assembly 270 to advance towards the first end 272 thereof, which in turn causes the struts 275 to bend and be radially deployed. Once the struts 275 are deployed, they are configured to radially support the first section 202 of the retriever 200. In some embodiments, where the second section 204 inverts and collapses within the first section 202, the second section 204 could overlay the reinforcement struts 275 within the interior portion 230 of the retriever 200, thereby sandwiching the reinforcement struts 275 between the first section 202 and the second section 204 of the mesh structure 175, as generally shown in FIG. 13.

Referring now to FIGS. 21I-K, there are shown various views of an alternative embodiment of a deployable reinforcement device 550. This embodiment is similar to the deployable reinforcement strut assembly 270 shown in FIGS. 21A-H and described above in that the reinforcement device 550 can reside within the interior of the retriever 200 and transition to a deployed configuration in response to the retriever 200 being transitioned to the deployed configuration. In this embodiment, the deployable reinforcement device 550 includes a reinforcement mesh structure 556 having a first end 552 and a second end 554. In one embodiment, the first end 552 can be coupled to the distal portion end of the first section 202 and the second end 554 can be coupled to the proximal portion of the second section 204. In another embodiment, one of the ends 552, 554 could be coupled to the corresponding section 202, 204 of the retriever mesh structure (not shown) and the other end could be unconnected or free floating, i.e., the deployable reinforcement device 550 could cantilever from one of the sections 202, 204 of the retriever mesh structure (not shown). In either embodiment, the deployable reinforcement device 550 is configured to move in conjunction with the movement of the corresponding sections 202, 204 of the retriever 200. The reinforcement mesh structure 556 of the deployable reinforcement device 550 is configured to expand and/or contract in response to the relative movement of the ends of the device 550. In particular, when the ends 552, 554 of the deployable reinforcement device 550 are extended from each other, as shown in FIG. 211, the reinforcement mesh structure 556 collapses into a relatively slim profile. Further, when the ends 552, 554 are advanced towards each other (e.g., via the transition of the retriever 200 from its collapsed configuration to the deployed configuration), the reinforcement mesh structure 556 of the deployable reinforcement device 550 correspondingly transitions to an intermediate configuration (FIG. 21J) and then a deployed configuration (FIG. 21K). In the deployed configuration, the reinforcement mesh structure 556 of the deployable reinforcement device 550 is configured to present a reinforcing edge 558 that is configured to bear against and radially support the retriever 200, which provides the corresponding benefits as noted above for the deployable reinforcement strut assembly 270.

When in the collapsed configuration shown in FIG. 211, the deployable reinforcement device 550 is sized, shaped, or otherwise configured such that it can reside within the collapsed interior of the retriever 200. In other words, in the collapsed configuration, the deployable reinforcement device 550 presents a slim enough profile such that it can reside within the interior volume of the retriever 200 without substantially impacting the profile of the retriever 200. When the retriever 200 is transitioned to the deployed configuration, the movement of the second section 204 towards the first section 202 correspondingly causes the second end 554 of the deployable reinforcement device 550 to advance towards the first end 552 thereof, which in turn causes the mesh structure to be radially deployed to define a reinforcing edge 558. The reinforcing edge 558 is configured to radially support the first section 202 of the retriever 200.

In some embodiments, the retriever 200 can include a helical support wire 280 (FIG. 22) extending throughout the length thereof. The helical support wire 280 can be configured to radially support the retriever 200 along all or a portion of its length. In one embodiment, the helical support wire 280 can be constructed from nitinol or other materials described herein. The helical support wire 280 can be affixed to the internal surface of the mesh structure 175.

In some embodiments, the mesh structure 175 of the retriever 200 could include a variety of different coatings, such as urethane or silicone. The coatings could be beneficial in order to facilitate attachment between reinforcing elements (e.g., the reinforcement struts 265, deployable reinforcement struts 275, or helical support wire 280).

### Retriever—Expandable

In some embodiments, retrievers for use with the aspiration systems described herein could be configured to transition between a first or collapsed configuration and a second or expanded configuration. In the collapsed configuration, the retriever is configured to be delivered through a catheter to the UIM for the removal thereof. In the expanded configuration, the retriever is configured to transition to a diameter that generally conforms to the diameter of the anatomical structure from which the UIM is being removed and collect the UIM in order to draw the UIM towards the distal end of the suction cannula (e.g., the section cannula 94 shown FIGS. 1, 7A, and 7B or the suction cannula 160 shown in FIG. 8A) for removal from the vessel and/or organ, in the manner as described above. Various embodiments of such retriever assemblies are shown in FIGS. 23-25B. In particular, FIG. 23 illustrates the retriever 300 including the shaft 310, FIG. 24 illustrates the mesh structure 352 of the retriever 300 absent any supporting components, and FIGS. 25A and 25B illustrate an embodiment of the retriever 300 including the supporting components or tethers 360.

As with the aforementioned embodiments, the retriever 300 can include a series of struts 350 (FIG. 24) that are interconnected and/or interwoven to define a mesh structure 352. The retriever 300 can transition between the collapsed configuration (such as is shown in FIG. 11) and an expanded configuration (FIGS. 22 and 23) due to the nature of the mesh structure 352 and the flexible materials from which the retriever 300 is fabricated, as described above in relation to the embodiments shown in FIGS. 9-22. When in the expanded configuration as shown in FIGS. 23-25B, this embodiment includes a body section 302 and an end 304 connected by a tapered section 306. The retriever 300 further includes a shaft 310 that extends through the interior 230 and is slidably movable through the end 304. In operation, one of the end 304 or the shaft 310 can be coupled to one of the movable portions of the delivery device (e.g., the first shaft 252 of the delivery device 250 in FIGS. 14A and 14B) such that the shaft 310 and the end 304 are movable with respect to each other. In other words, in various embodiments, one of the end 304 or the shaft 310 is movable with respect to the corresponding component. Accordingly, the retriever 300 can be transitioned from the collapsed configuration to the expanded configuration by actuating one of the movable portions of the delivery device (once again, e.g., the second shaft 254), which causes the mesh structure 352 of the retriever 300 to collapse when the end 304 is extended distally or expand when the end 304 is pulled proximally. The various dimensions and/or parameters of the body section 302, end 304, and/or tapered section 306 can be the same or similar to the dimensions described above and set forth in TABLE 2 for the aforementioned embodiments. The body section 302 defines a leading edge 320, which in turn defines a semi-spherical open interior 330 that is configured to collect the

In using aspiration systems (e.g., the aspiration system 10 shown in FIGS. 1-7B or the aspiration system 150 shown in FIGS. 8A and 8B) with the retriever 300, the subject and aspiration system components are prepared as generally described above. After the initial preparation, the targeted anatomical structure (e.g., a vessel 168) is accessed. Accordingly, the retriever 300 is advanced (e.g., via the ancillary port 88 of the aspiration device 12) in the collapsed configuration through cannula or catheter, such as the suction cannula 94 (FIG. 1) or the suction cannula 160 (FIG. 8A), toward the targeted UIM in the anatomical structure. In some embodiments, the user can monitor the advancement of the retriever 300 under fluoroscopic imaging or via another imaging modality. As the retriever 300 is advanced towards the UIM, it exits the catheter or cannula, expanding from the collapsed configuration (such as shown in FIG. 11) to the expanded configuration (FIGS. 23-25B). In various embodiments, the retriever 300 can be configured to automatically unfurl to its expanded configuration or unfurl to the expanded configuration in response to manual actuation by the operator, e.g., via the handle assembly 255. Once expanded, the retriever 300 can be advanced towards the UIM, thereby causing the UIM to be mechanically disrupted and/or collected by the expanded retriever 300. In some embodiments, after collecting the UIM within the retriever 300, the retriever 300 can be drawn towards the suction cannula 94 to cause the UIM to become engaged therewith (such as is shown in FIGS. 16A and 16B). In some embodiments, as the retriever 300 is retracted, the leading edge 320 of the expanded retriever 300 can further be dragged along the internal surface of the target anatomical structure, dislodging the UIM from the walls of the anatomical structure and/or removing any additional or excess UIM therefrom. Further, the aspiration system can be activated to remove the captured and/or mechanically disrupted UIM from the target anatomical structure. Once the UIM has been removed from the subject, the aspiration system and its various components, including the retriever assembly, can accordingly be removed from the subj ect.

As shown in FIGS. 25A and 25B, in some embodiments, the retriever 300 can further include a series of tethers 260 connecting the leading edge 320 of the mesh structure 352 to the shaft extending therethrough. In this embodiment, the retriever 300 can define a parachute-like shape or configuration. The tethers 360 can beneficial because they could assist in reinforcing or supporting the leading edge 320 when the retriever 300 is in the expanded configuration, as shown in FIGS. 25A and 25B. Additionally, the tethers 360 assist in expansion of the retriever 300 by coupling the retriever 300 to the shaft which is in turn controlled by the handle assembly to transition the retriever 300 between collapsed and expanded configurations.

Referring now to FIGS. 26A-28, in some embodiments, the retriever 300 can further include a reinforcing element 500 that is configured to reinforce the leading edge 320 of the retriever 300. The reinforcing element 500 include a wire 502 that is arranged in a helical manner to define a circular shape that corresponds to the leading edge 320 defining the interior portion 330 of the retriever 300. The wire 502 could be fabricated from a variety of different materials, including, e.g., nitinol or another shape-memory material. The reinforcing element 500 can be configured to stiffen and/or radially support the leading edge 320 in order to support and improve the ability of the leading edge 320 to mechanically disrupt or dislodge the UIM from the interior surface of the anatomical structure.

As noted above, in some applications, the leading edge 320 can be dragged along the interior wall of the anatomical structure to assist in the removal of the UIM. Accordingly, it is beneficial for the leading edge 320 to be designed to atraumatically treat the anatomical structure. Therefore, it would be beneficial for the reinforcing element 500 to be joined to the mesh structure 352 of the leading edge 320 such that it presents an atraumatic surface for treatment of the anatomical structure. In one embodiment, the wire 502 can include a connector 504 including a helical structure 506 (e.g., fabricated from nitinol) around the wire 502, which includes a loop 508. The wire 502 of the reinforcing element can include one or multiple connectors 504, as shown in FIG. 26F. The loops 508 can include, for example, U-shaped loops 508A (FIG. 26C), V-shaped loops 508B (FIG. 26D), or twisted loops 508C (FIG. 26E). The loops 508 are looped or tied into corresponding loops 520 (FIG. 26G) at the distal end of the leading edge 320 of the mesh structure 352. Because the reinforcing element 500 is tied into the mesh structure 320 of the leading edge 320, it does not present any sharp surfaces or protrusions that project therefrom and would present a traumatic edge unsuitable for treating the interior walls of an anatomical structure. In one embodiment, the connectors 504 can further include slidable stops or attachment portions that are configured to provide the connectors with a partially constrained degree of movement along the length of the wire 502, thereby allowing the connectors 504 to shift in response to the changing geometry of the retriever 300 as it shifts between the various configurations, without providing fully unconstrained movement.

When the retriever 300 is in the expanded configuration shown in FIG. 27A, the reinforcing element 500 extends about and supports the leading edge 320 of the retriever 300. As the retriever 300 transitions to the collapsed configuration from the expanded configuration, the reinforcing element 500 is configured to unspool and transition to a generally linear, collapsed state that presents a streamlined profile, as shown in FIGS. 27B-D (with the retriever 300 removed for clarity).

Referring now to FIG. 28, in some embodiments the tethers 360 of the retriever 300 can be coupled to the reinforcing element 500 at the connectors 504. This embodiment can be beneficial because it allows for the connectors 504 to be utilized to attach the tethers 360 to the mesh structure 352 of the retriever 300, without requiring additional attachment points that would necessitate further manufacturing fabrication techniques.

In some embodiments, the retriever 300 can further include a spring section 260 (FIG. 18), reinforcement struts 265, 275 (FIGS. 19-20E), a helical support wire 280 (FIG. 21), coatings, or any other feature described above in connection with other embodiments that is not mutually exclusive thereto.

### Retriever—Helical

In some embodiments, retrievers for use with the aspiration systems 10 described herein could include retrievers that are adapted to frictionally engage with the UIM in order to facilitate the en bloc removal thereof. One embodiment of such a retriever assembly is shown in FIG. 29. In the depicted embodiment, the retriever 400 defines a helical or sugar-shaped mesh structure 452. As with the aforementioned embodiments, the retriever 400 can include a series of struts 450 that are interconnected and/or interwoven to define a mesh structure 452. In this embodiment, the mesh structure 452 defines a helical or auger-like shape, which includes a distal section 402 that terminates at a tapered point and a helical body section 404.

In this embodiment, the helical retriever 400 is delivered through a catheter to the UIM. After reaching the UIM, the helical retriever 400 can be longitudinally driven to pierce the UIM with the distal section 402. Once the distal section 402 is engaged with the UIM, a rotational force can be applied to the helical retriever 400 via the delivery device 250, thereby causing the helical body section 404 of the retriever 400 to be rotationally driven into and through the UIM (i.e., “screwed” into the UIM). Once the helical body section 404 is engaged with the UIM, the helical retriever 400 can be longitudinally retracted towards the distal end of the suction cannula 94 (FIG. 1) for removal from the vessel and/or organ, as described above. Once again, this process can be performed under fluoroscopic or other imaging techniques in order to monitor the location of the retriever 400, determine when the retriever 400 is engaged with the UIM, determine when to pull the retriever 400 towards the suction cannula 94, and so on.

In some embodiments, the retriever 300 can further include a spring section 260 (FIG. 18), reinforcement struts 265, 275 (FIGS. 19-20E), a helical support wire 280 (FIG. 21), or another other feature described above in connection with other embodiments that is not mutually exclusive thereto.

### Methods of Using the Retriever Assemblies

The various embodiments of retrievers 200, 300, 400 described herein can be utilized in a variety of different manners and/or with different techniques in order to grasp, collect, macerate, dislodge, or otherwise mechanically disrupt a UIM. In combination with the various embodiments of aspiration systems 10, 150, the retrievers 200, 300, 400 can accordingly be used to remove the UIM from the subject. Various processes that can be performed by clinician teams using the retrievers 200, 300, 400 and aspiration systems 10, 150 are described below. The various processes described below can be performed under a medical imaging modality (e.g., fluoroscopy) to allow the clinician team to monitor the performance of the steps of the processes.

Referring now to FIG. 30, there is shown a flow diagram of a process 700 for utilizing the various embodiments of the expandable retrievers 200, 300 described herein for collecting a UIM. In particular, the retriever is advanced 702 through the suction cannula (e.g., the suction cannula 94) and out of the funnel (e.g., the funnel 104) of the aspiration system (e.g., the aspiration system 10 or the aspiration system 150) and passed 704 through the UIM in its collapsed state. In other words, the retriever can be advanced distally away from the aspiration system into the anatomical structure. Because the retriever presents a slim profile when in its collapsed state (e.g., as shown in FIG. 11), it is able to pass through the UIM without mechanically disrupting or otherwise causing substantial fragmentation of the UIM. Further, the retriever is transitioned 706 from its collapsed state to the expanded state and transitioned 708 from its expanded state to its deployed state. In some embodiments, the clinicians can utilize the handle assembly 255 to control the transitioning 706, 708 of the retriever between the collapsed, expanded, and deployed states. In some embodiments, the transition from the collapsed state to the deployed state can be a single step process, whereas in other embodiments it can be a multistep process. In some embodiments, such as with the retriever 300 described in connection with FIGS. 23-28, the retriever can be configured to transition directly to the expanded or deployed configuration (FIGS. 23-25B) without an intermediate state. Once in the deployed state, the deployed retriever can be retracted 710 (i.e., advanced proximally with respect to the aspiration system) back towards the funnel to capture the UIM within the open interior 230 of the deployed retriever and draw the UIM towards the suction cannula, such as is shown in FIGS. 16A and 16B. Once the UIM has been brought adjacent to the suction cannula, once again as is shown in FIGS. 16A and 16B, the aspiration system can be activated 712 to remove the UIM from the subject and anatomical structure.

Referring now to FIG. 31, there is shown a flow diagram of a process 750 for utilizing the various embodiments of the expandable retrievers 200,300 described herein for mechanically disrupting or dislodging a UIM. This process 750 can begin similarly to the process 700 described above. In particular, the retriever is advanced 702 through the suction cannula (e.g., the suction cannula 94) and out of the funnel (e.g., the funnel 104) of the aspiration system (e.g., the aspiration system 10 or the aspiration system 150) and passed 704 through the UIM in its collapsed state. Further, the retriever is transitioned 706 from its collapsed state to the expanded state and transitioned 708 from its expanded state to its deployed state. As with the process 700 described in connection with FIG. 30, clinicians can utilize the handle assembly 255 to control the transitioning 706,708 of the retriever between the various states and the transition from the collapsed state to the deployed state could be a single step or multistep process. Once in the deployed state, the deployed retriever can be retracted 710 (i.e., advanced proximally with respect to the aspiration system) back towards the funnel to capture the UIM within the open interior 230 of the deployed retriever and draw the UIM towards the suction cannula, such as is shown in FIGS. 16A and 16B. However, this process 750 deviates from the process 700 shown in FIG. 30 in that once the UIM has been brought adjacent to the suction cannula, once again as is shown in FIGS. 16A and 16B, the retriever is further retracted 752 into the funnel, thereby causing the funnel to effectively function as a procedural sheath (such as the procedural sheath 126 described above in connection with FIGS. 1-7B) that squeezes the retriever, thereby causing the retriever to partially collapse and force the UIM into the funnel and/or the lumen of the suction cannula to disrupt or dislodge the UIM from the anatomical structure. Alternatively, the clinician team could actuate the retriever (e.g., via the handle assembly 255) to force the retriever into the partially collapsed configuration and thereby drawn the UIM into the funnel and/or suction cannula. The aspiration system could further be activated to remove the UIM from the subject and anatomical structure.

Referring now to FIG. 32, there is shown a process 800 for using the expandable retrievers 200, 300 in combination with a continuous recirculation aspiration system, such as the aspiration system 150 described above in connection with FIGS. 8A and 8B. This process 800 can begin similarly to the process 700 described above. In particular, the retriever is advanced 702 through the suction cannula (e.g., the suction cannula 160) and out of the funnel (e.g., the distal end 11 of cannula 160) of the aspiration system 150 and passed 704 through the UIM in its collapsed state. Further, the retriever is transitioned 706 from its collapsed state to the expanded state and transitioned 708 from its expanded state to its deployed state. As with the process 700 described in connection with FIG. 30, clinicians can utilize the handle assembly 255 to control the transitioning 706, 708 of the retriever between the various states and the transition from the collapsed state to the deployed state could be a single step or multistep process. Once in the deployed state, the deployed retriever can be retracted 802 (i.e., advanced proximally with respect to the aspiration system) back towards the distal end of the suction cannula in a manner that mechanically disrupts the UIM. For example, the deployed retriever could be pushed or pulled against the UIM to mechanically disrupt the UIM and dislodge it from the interior walls of the anatomical structure. In some applications, the edges of the retriever could additionally be atraumatically dragged along the interior walls of the anatomical structure to facilitate the dislodgment of the UIM therefrom. In conjunction with the mechanical disruption of the the aspiration system 150 can be utilized to apply 804 a continuous suction via the suction cannula 160, thereby collecting the mechanically disrupted UIM and/or fragments thereof. The fluid collected and filtered via the aspiration system 150 could further be reinfused into the subject, as described above.

In other embodiments, the helical retriever 400 described in connection with FIG. 29 could be used similarly to the processes described above. In particular, the helical retriever 400 could be utilized to bring a UIM adjacently to a suction cannula (as in the process 700 in FIG. 30), draw the UIM into a suction cannula (as in the process 750 in FIG. 31), or mechanically disrupt the UIM (as in the process 800 in FIG. 32). The difference in the process implemented utilizing the helical retriever 400 is that the helical retriever is not passed through the UIM and then transitioned into a deployed configuration; rather, the helical retriever 400 is advanced into the UIM (e.g., via the combination of longitudinal and rotational movement) to mechanically engage the helical retriever 400 with the UIM. Alternatively, the helical retriever 400 is passed through the UIM in the collapsed configuration and expanded once past the UIM. As the helical retriever 400 is retracted towards the funnel, the helical retriever 200 can be rotated such the combination of longitudinal and rotational movement mechanically engages the UIM. Once the helical retriever 400 is mechanically engaged with the UIM, the UIM can then be removed from the anatomical structure in accordance with the remaining steps of the processes 700, 750, 800 described above.

It should be noted that the processes described above are not limited to any particular directionality with respect to the subject and/or anatomical structures. For example, in applications involving treatment of UIM within vessels, the processes could include inserting the retrievers upstream or downstream of the UIM and advancing the retriever accordingly towards the UIM. For example, in some applications it could be beneficial to access a vessel upstream of the UIM and advance the retriever downstream to the UIM in order to mechanically disrupt the UIM and cause the fragments to flow downstream to the suction cannula of the aspiration system for removal. In other applications, it could be beneficial to access a vessel downstream of the UIM and advance the retriever upstream to the UIM in order to collect and/or mechanically disrupt the UIM and guide the UIM (either en bloc or in fragments) back to the suction cannula for removal.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A retriever for use with an aspiration system for removal of undesirable intravascular material (UIM), the aspiration system comprising a catheter, the retriever comprising:
a plurality of struts that are interconnected to define a mesh structure, the mesh structure comprising a first section and a second section connected at an inversion point;
wherein:
the mesh structure is configured to transition between a collapsed configuration, an expanded configuration, and a deployed configuration,
in the collapsed configuration, the mesh structure comprises an outer diameter less than or equal to an internal diameter of a lumen of the catheter such that the mesh structure is configured to be movable through the lumen,
the mesh structure is configured to transition from the collapsed configuration to the expanded configuration in response to exiting the catheter,
in the expanded configuration, the outer diameter of the mesh structure is greater than the outer diameter in the collapsed configuration,
in the expanded configuration, the mesh structure is configured such that application of a longitudinal force thereto causes the second section to invert at the inversion point and collapse into the first section, thereby forming a semi-spherical shape defining the deployed configuration, and
the semi-spherical shape is configured to receive and hold the

2. The retriever of claim 1, wherein the outer diameter is from about 8 mm to about 20 mm.

3. The retriever of claim 1, wherein the outer diameter corresponds to a diameter of an anatomical structure from which the UIM is being removed.

4. The retriever of claim 1, further comprising:
one or more reinforcement struts disposed within the first section, the one or more reinforcement configured to radially support the first section.

5. The retriever of claim 1, wherein the plurality of struts comprises a shape-memory material.

6. The retriever of claim 5, wherein the shape-memory material comprises nitinol.

7. The retriever of claim 5, wherein the shape of the deployed configuration has been heat set.

8. A retriever assembly system for use with an aspiration system for removal of undesirable intravascular material (UIM), the retriever assembly comprising:
a catheter delivery device comprising:
a catheter comprising a lumen, and
an outer sheath configured to be moved longitudinally with respect to the catheter; and
a retriever comprising a plurality of struts that are interconnected to define a mesh structure, the mesh structure comprising a first section and a second section connected at an inversion point;
wherein:
the mesh structure is configured to transition between a collapsed configuration, an expanded configuration, and a deployed configuration,
in the collapsed configuration, the mesh structure comprises an outer diameter less than or equal to an internal diameter of a lumen of the catheter such that the mesh structure is configured to be movable through the lumen,
the mesh structure is configured to transition from the collapsed configuration to the expanded configuration in response to exiting the catheter,
in the expanded configuration, the outer diameter of the mesh structure is greater than the outer diameter in the collapsed configuration,
in the expanded configuration, the mesh structure is configured such that application of a longitudinal force thereto causes the second section to invert at the inversion point and collapse into the first section, thereby forming a semi-spherical shape defining the deployed configuration, and
the semi-spherical shape is configured to receive and hold the

9. The retriever assembly system of claim 8, wherein the outer diameter is from about 8 mm to about 20 mm.

10. The retriever assembly system of claim 8, wherein the outer diameter corresponds to a diameter of an anatomical structure from which the UIM is being removed.

11. The retriever assembly system of claim 8, wherein the retriever further comprises one or more reinforcement struts disposed within the first section, the one or more reinforcement configured to radially support the first section.

12. The retriever assembly system of claim 8, wherein the plurality of struts comprises a shape-memory material.

13. The retriever assembly system of claim 12, wherein the shape-memory material comprises nitinol.

14. The retriever assembly system of claim 12, wherein the shape of the deployed configuration has been heat set.

15. A method for removing undesirable intravascular material (UIM) from an anatomical structure of a subject via an aspiration system, the method comprising:
accessing the anatomical structure with a retriever assembly, the retriever assembly comprising:
a catheter delivery device comprising:
a catheter comprising a lumen, and an outer sheath configured to be moved longitudinally with respect to the catheter, and
a retriever comprising a plurality of struts that are interconnected to define a mesh structure, the mesh structure comprising a first section and a second section connected at an inversion point, wherein the mesh structure is configured to transition between a collapsed configuration, an expanded configuration, and a deployed configuration;
advancing the retriever in the collapsed state, via the catheter past the UIM, thereby causing the retriever to exit the catheter upstream of the UIM and transition to the expanded configuration, wherein in the expanded configuration, the outer diameter of the mesh structure is greater than the outer diameter in the collapsed configuration;
applying, via the outer sheath, a longitudinal force to the retriever, thereby causing the second section to invert at the inversion point and collapse into the first section and forming a semi-spherical shape defining the deployed configuration; and
distally translating the retriever in the deployed configuration to cause the retriever to collect the UIM and draw the UIM towards a suction source of the aspiration system.

16. The method of claim 15, wherein the outer diameter is from about 8 mm to about 20 mm.

17. The method of claim 15, wherein the outer diameter corresponds to a diameter of an anatomical structure from which the UIM is being removed.

18. The method of claim 15, wherein the retriever further comprises one or more reinforcement struts disposed within the first section, the one or more reinforcement configured to radially support the first section.

19. The method of claim 15, wherein the plurality of struts comprises a shape-memory material.

20. The method of claim 19, wherein the shape-memory material comprises nitinol.
